SEQUENCE LISTING (4 sequences)

SEQ ID NO: 1 - DNA, 49 bp, Artificial Sequence
  Synthetically generated primer
gcagctccataggattggtcgacggcattctgatgatgaggtccgcttg

SEQ ID NO: 2 - DNA, 40 bp, Artificial Sequence
  Synthetically generated primer
atacatctgcaggcttcaagaataggcatgaggctttctc

SEQ ID NO: 3 - DNA, 40 bp, Artificial Sequence
  Synthetically generated primer
ttctgctctagatgtagtactttgggctatttcggctgtt

SEQ ID NO: 4 - DNA, 47 bp, Artificial Sequence
  Synthetically generated primer
ctgataccactcctatggaaccgctggatgatcagaaagcagttcag

(19) United States
(12) Patent Application Publication
(10) Pub. No.: US 2009/0269832 A1
(43) Pub. Date: Oct. 29, 2009

(54) Growth of Microorganisms in Media Containing Crude Glycerol

(75) Inventors: Kevin A. Jarrell, Lincoln, MA (US); Gabriel Reznik, Waltham, MA (US)

Correspondence Address: CHOATE, HALL & STEWART LLP, TWO INTERNATIONAL PLACE, BOSTON, MA, 02110, US

(73) Assignee: Modular Genetics, Inc., Cambridge, MA (US)

(21) Appl. No.: 12/264,491

(22) Filed: Nov. 4, 2008

Related U.S. Application Data
(60) Provisional application No. 60/985,480, filed on Nov. 5, 2007.

(51) Int. Cl. C12N 1/20; C12N 1/16

(52) U.S. Cl. 435/252.5; 435/253.6; 435/255.21

(57) ABSTRACT

The present invention provides novel methods of growing of microorganisms in cell culture media comprising crude glycerol as a carbon source. The present invention further provides novel cell culture media comprising crude glycerol as a carbon source. In certain embodiments, inventive cell culture media substantially lack refined glycerol. In certain embodiments, inventive cell culture media comprise crude glycerol as the sole carbon source.

### CROSS REFERENCE TO RELATED APPLICATIONS

This application is copending with, shares at least one common inventor with, and claims priority to U.S. provisional patent application No. 60/985,480, filed Nov. 5, 2007. The prior application is hereby incorporated by reference in its entirety.

This application contains a Sequence Listing submitted as an electronic text file named “Seq_Listing_ST25.txt”, having a size in bytes of 1085, and created on Oct. 28, 2008. The information contained in this electronic file is hereby incorporated by reference in its entirety.

### BACKGROUND

Microorganisms are typically grown in cell culture media that contain a carbon source. Carbon sources are often simple sugars such as glucose or galactose, which are broken down and converted to energy, cellular components, and/or metabolic products. The choice of which carbon source to use in the culturing of microorganisms is determined by a variety of factors including considerations such as the ability of the microorganism to utilize a particular carbon source, the ability of the microorganism to convert a particular carbon source into a product of interest, the type and amount of byproducts produced as a result of metabolizing the carbon source, the availability of a carbon source, the present and/or future cost a particular carbon source, etc.

In some cases, microorganisms are grown in cell culture media that contain refined glycerol as an energy source. As a main by-product of biodiesel production, raw or crude glycerol (also called glycerin) is accumulating at increasing rates with the advent of large scale commercialization. Refined glycerol is typically generated from crude glycerol through an intensive process that removes contaminants and impurities that are generally thought to be detrimental to the growth of microorganisms. As a result, refined glycerol is relatively expensive and time-consuming to produce, and is thus not a favored carbon source for commercial-scale growth of microorganisms and/or for the commercial-scale production of compounds produced by such microorganisms.

### SUMMARY OF THE INVENTION

The present invention provides improved compositions and methods for growing microorganisms in cell culture media using carbon sources that traditionally were considered unsuitable and/or undesirable for microbial growth. In certain embodiments, methods are provided wherein a microorganism is grown in a cell culture comprising crude glycerol as an energy source. In certain embodiments, methods are provided wherein a microorganism is grown in a cell culture comprising crude glycerol as an energy source, which cell culture further substantially lacks refined glycerol. In certain embodiments, methods are provided wherein a microorganism is grown in a cell culture comprising crude glycerol as the sole energy source.

The present invention also provides improved culture media suitable for growth of microorganisms. In certain embodiments, a cell culture medium of the present invention comprises crude glycerol as an energy source. In certain embodiments, a cell culture medium of the present invention comprises crude glycerol as an energy source, which cell culture medium further substantially lacks refined glycerol. In certain embodiments, a cell culture medium of the present invention comprises crude glycerol as the sole energy source.

Any of a wide variety of microorganisms can be grown in inventive cell culture media that comprise crude glycerol as an energy source. For example, any of a variety of bacteria may be grown according to the present invention. As non-limiting examples, bacteria of the genera Bacillus, Clostridium, Enterobacter, Klebsiella, Micromonospora, Actinoplanes, Dactylosporangium, Streptomyces, Kitasatospora, Amycolatopsis, Saccharopolyspora, Saccharothrix and Actinosynnema may be grown in accordance with compositions and/or methods of the present invention. In certain embodiments, a bacterium of the genus Bacillus grown is grown in accordance with compositions and/or methods of the present invention. In certain embodiments, a bacterium of the species Bacillus subtilis is grown in accordance with compositions and/or methods of the present invention.

Additionally or alternatively, any of a variety of fungi may be grown according to the present invention. In certain embodiments, a fungus grown in accordance with compositions and/or methods of the present invention is a yeast. As non-limiting examples, yeast of the genera Saccharomyces, Pichia, Aspergillus, Trichoderma, Kluyveromyces, Candida, Hansenula, Schizpsaccaromyces, Yarrowia, Chrysoporium, Rhizopus, Aspergillus and Neurospora may be grown in accordance with compositions and/or methods of the present invention. In certain embodiments, a yeast of the genus Saccharomyces grown is grown in accordance with compositions and/or methods of the present invention. In certain embodiments, a yeast of the species Saccharomyces cerevisiae is grown in accordance with compositions and/or methods of the present invention.

In certain embodiments, a microorganism grown in an inventive cell culture medium and/or according to inventive methods produces a polypeptide, non-ribosomal peptide, and/or lipopeptide of interest. As one non-limiting example, a microorganism grown in an inventive cell culture medium and/or according to inventive methods may produce surfactin. Those of ordinary skill in the art will be aware of other polypeptides, non-ribosomal peptides, and/or lipopeptides of interest, as well as microorganisms that produce them. Such art-recognized polypeptides, non-ribosomal peptides, and/or lipopeptides of interest can be grown in inventive cell culture media and/or according to methods of the present invention. In certain embodiments, such a microorganism produces the polypeptide, non-ribosomal peptide, and/or lipopeptide of interest to a level that is at least that of a microorganism grown in traditional cell culture media and/or according to traditional methods. In certain embodiments, the yield (defined as percent of carbon source converted into a product of interest) of a polypeptide, non-ribosomal peptide, and/or lipopeptide of interest produced by a microorganism grown in inventive media containing crude glycerol is at least about 10%, 15%, 20%, 25%, 30%, 35%, 40%, 45%, 50%, 55%, 56%, 57%, 58%, 59% 60% or more.

In certain embodiments, a microorganism grown in an inventive cell culture medium and/or according to inventive methods that produces a polypeptide, non-ribosomal peptide, and/or lipopeptide of interest is a bacterium. As non-limiting examples, bacteria of the genera Bacillus, Clostridium, Enterobacter, Klebsiella, Micromonospora, Actinoplanes, Dactylosporangium, Streptomyces, Kitasatospora, Amycolatopsis, Saccharopolyspora, Saccharothrix and Actinosynnema may be grown in accordance with compositions and/or methods of the present invention to produce a polypeptide, non-ribosomal peptide and/or lipopeptide of interest. In certain embodiments, such a bacterium is of the genus Bacillus. In certain embodiments, such a bacterium is of the species Bacillus subtilis.

In certain embodiments, methods are provided wherein production of a polypeptide, non-ribosomal peptide, and/or lipopeptide of interest is increased by adding tryptone, total soy extract, yeast extract, casamino acids and/or distiller grains to the cell culture after initiation. In certain embodiments, a nitrogen source is added after the cell culture has reached stationary phase. In certain embodiments, a nitrogen source is added while the cell culture is in growth phase. Nitrogen sources that can be used in accordance with the present invention include, but are not limited to, tryptone, total soy extract, yeast extract, casamino acids and/or distiller grains. In certain embodiments, a microorganism produces a polypeptide, non-ribosomal peptide, and/or lipopeptide of interest to an increased level relative to the level of polypeptide, non-ribosomal peptide, and/or lipopeptide that would be produced by a microorganism grown under otherwise identical conditions in an otherwise identical cell culture medium that lacks the provided nitrogen source. In certain embodiments, a polypeptide, non-ribosomal peptide, and/or lipopeptide of interest produced in a cell culture to which a nitrogen source is added represents an increased fraction of the total biomass of the cell culture compared the fraction that would result if the nitrogen source were not added to the cell culture.

In certain embodiments, a microorganism grown in an inventive cell culture medium and/or according to inventive methods produces an organic acid of interest. As non-limiting examples, a microorganism grown in an inventive cell culture medium and/or according to inventive methods may produce pyruvic acid, succinic acid, fumaric acid, malic acid, maleic acid and/or citric acid. Those of ordinary skill in the art will be aware of other organic acids of interest, as well as microorganisms that produce them. Such art-recognized organic acids of interest can be grown in inventive cell culture media and/or according to methods of the present invention. In certain embodiments, such a microorganism produces the organic acid of interest to a level that is at least that of a microorganism grown in traditional cell culture media and/or according to traditional methods. In certain embodiments, the yield of an organic acid of interest produced by a microorganism grown in inventive media containing crude glycerol is at least about 10%, 15%, 20%, 25%, 30%, 35%, 40%, 45%, 50%, 55%, 56%, 57%, 58%, 59%, 60%, 65%, 70%, 75%, 80%, 85%, 90% or more.

In certain embodiments, a microorganism grown in an inventive cell culture medium and/or according to inventive methods that produces an organic acid of interest is a fungus. In certain embodiments, a fungus grown in accordance with compositions and/or methods of the present invention is a yeast. As non-limiting examples, yeast of the genera Saccharomyces, Pichia, Aspergillus, Trichoderma, Kluyveromyces, Candida, Hansenula, Schizpsaccaromyces, Yarrowia, Chrysoporium, Rhizopus, Aspergillus and Neurospora may be grown in accordance with compositions and/or methods of the present invention to produce an organic acid of interest. In certain embodiments, such a yeast is of the genus Saccharomyces. In certain embodiments, such a yeast is of the species Saccharomyces cerevisiae.

### BRIEF DESCRIPTION OF THE DRAWING

FIGS. 1A-1D show growth curves (OD650 nm/10) vs. time using various soy extract concentrations and 0.5% refined glycerol (FIG. 1A, 0.1% soy extract; FIG. 1B., 0.25% soy extract; FIG. 1C, 0.5% soy extract; FIG. 1D, 1% soy extract).

FIG. 2 shows a growth curve of Bacillus subtilis in M9YE with 1% soy extract+various concentrations of glycerol.

FIG. 3 shows a comparison of hemolytic activity of OKB105 D(upp)Spect^{R} cells grown in media containing either refined glycerol or crude glycerol. The top sample was grown in M9YE+glucose (5 g/L) +tryptone (40 g/L). Row 1=0.25% soy & 0.25% glycerol. Row 2=0.25% soy & 0.50% glycerol. Row 3=1% soy & 0.25% glycerol. Row 4=1% soy & 0.50% glycerol. Column 1=Crude glycerol with 2% soy extract supplementation. Column 2=Crude glycerol with 4% soy extract supplementation. Column 3=Clean glycerol with 2% soy extract supplementation. Column 4=Clean glycerol with 4% soy extract supplementation.

FIG. 4 shows a comparison of hemolytic activity of OKB105 D(upp)Spect^{R} cells grown in media containing either glucose or crude glycerol.

FIG. 5 shows hemolytic activity of the samples described in Table 2. Numbering begins at the top left to right and proceeds left to right, top to bottom. Thus, the top left spot is sample 22916, while the bottom right spot is sample 22943. The largest halos were obtained by supplementation of M9YE with ˜0.3% soy protein and 0.5%, 1%, and 2% glycerol.

FIG. 6 shows m/z peaks for the determination of surfactin titer for sample 22939 described in Table 2.

FIG. 7 shows Surfactin titer using Pr, a temperature inducible promoter from bacteriophage lambda.

FIG. 8 shows a time course of S. cerevisiae growth in glucose and glycerol-supplemented media. Independent cultures were initiated in YP media supplemented with glucose, purified glycerol, raw glycerol, or no sugar. Growth was monitored by spectrophotometry until saturation was reached.

FIG. 9 shows a time course of Saccharomyces cerevisiae growth in glucose and glycerol-supplemented media. Independent cultures initiated in nitrogen-limited acid production media supplemented with glucose or raw glycerol were monitored by spectrophotometry and hemocytometry until saturation was reached.

FIG. 10 shows hemolytic analyses for surfactin produced by the seven samples Example 6, Table 7. Spot 1 is minimal salts+33 g/L distillers grains+170 g/L maltose (filtered solution. Spot 2 is minimal salts+33 g/L distillers grains+85 g/L maltose (filtered solution). Spot 3 is minimal salts+33 g/L distillers grains+170 g/L maltose (autoclaved in media). Spot 4 is minimal salts+33 g/L distillers grains+85 g/L maltose (autoclaved in media). Spot 5 is minimal salts+66 g/L distillers grains+170 g/L maltose (filtered solution). Spot 6 is minimal salts+66 g/L distillers grains+85 g/L maltose (filtered solution). Spot 7 is minimal salts+33 g/L distillers grains+5 g/L refined glycerol (filtered solution).

FIG. 11 shows Maldi data from the seven samples Example 6, Table 7. Samples are top to bottom, samples 7, 6, 5, 4, 3, 2, 1 as depicted in FIG. 10.

FIG. 12 shows hemolytic analyses for temperature shift experiments using minimal salts media and 5 g/L crude glycerol with 10 g/L tryptone or 33 g/L distillers grains supplementation. Rows 1-3: Cells grown to OD650˜0.5 at 37° C., supplemented immediately with tryptone (day 2=column 1, day 3=column 2), distillers grains (day 2=column 3, day 3=column 4), or unsupplemented for 3 days=column 5, and shifted to 30° C. (row1), 37° C. (row2) or 45° C. (row3). Row 4: Cells grown to OD650˜0.5 at 45° C., supplemented immediately with tryptone (day 2=column 1, day 3=column 2), distillers grains (day 2=column 3, day 3=column 4), or unsupplemented for 3 days=column 5, and returned to 45° C.

### DETAILED DESCRIPTION OF CERTAIN EMBODIMENTS

### Definitions

“Crude glycerol”: The term “crude glycerol” as used herein refers to glycerol that has not been subjected to art-recognized processes that remove contaminants and/or impurities to generate “refined glycerol” (see definition of “refined glycerol”, infra). Crude glycerol is produced by a variety of natural and synthetic processes. For example, crude glycerol is produced during the process of biodiesel production. Additionally, crude glycerol is produced during the process of saponification (e.g., making soap or candles from oils or fats). Crude glycerol of the present invention may be subjected to one or more processes to render it suitable and/or more advantageous for use in growing microorganisms without converting it to “refined glycerol” as the term is used herein. For example, crude glycerol for use in compositions and methods of the present invention may be autoclaved to sterilize it. Additionally or alternatively, crude glycerol for use in compositions and methods of the present invention may be subjected to a filtration step to remove solids and other large masses. Such filtration can be performed on crude glycerol itself of on a culture medium that comprises crude glycerol. Crude glycerol subjected to such processes is not “refined glycerol” as the term is used herein.

“Lipopeptide”: The term “lipopeptide” as used herein refers to any of a variety of molecules that contain a peptide backbone covalently linked to one or more fatty acid chains. Often, lipopeptides are produced naturally by certain microorganisms. In such cases, a lipopeptide is typically produced by one or more nonribosomal peptide synthetases that build an amino acid chain without reliance on the canonical translation machinery. For example, surfactin is cyclic lipopeptide that is naturally produced by certain bacteria, including the Gram-positive endospore-forming bacteria Bacillus subtilis. Surfactin consists of a seven amino acid peptide loop, and a hydrophobic fatty acid chain (beta-hydroxy myristic acid) thirteen to fifteen carbons long. The fatty acid chain allows permits surfactin to penetrate cellular membranes. The peptide loop is composed of the amino acids glutamic acid, leucine, D-leucine, valine, aspartic acid, D-leucine and leucine. Glutamic acid and aspartic acid residues at positions 1 and 5 respectively, constitute a minor polar domain. On the opposite side, valine residue at position 4 extends down facing the fatty acid chain, making up a major hydrophobic domain. Surfactin is synthesized by the linear nonribosomal peptide synthetase, surfactin synthetase is synthesized by the three surfactin synthetase subunits SrfA-A, SrfA-B, and SrfA-C. Each of the enzymes SrfA-A and SrfA-B consist of three amino acid activating modules, while the monomodular subunit SrfA-C adds the last amino acid residue to the heptapeptide. Additionally the SrfA-C subunit includes the thioesterase domain (“TE domain”), which catalyzes the release of the product via a nucleophilic attack of the beta-hydroxy of the fatty acid on the carbonyl of the C-terminal Leu of the peptide, cyclizing the molecule via formation of an ester. Other lipopeptides and their amino acid and fatty acid compositions are known in the art, and can be produced in accordance with compositions and/or methods of the present invention.

“Non-ribosomal peptide”: The term “non-ribosomal peptide” as used herein refers to a peptide chain produced by one or more nonribosomal peptide synthetases. Thus, as opposed to “polypeptides” (see definition, infra), non-ribosomal peptides are not produced by a cell's ribosomal translation machinery. Polypeptides produced by such nonribosomal peptide synthetases may be linear, cyclic or branched. Numerous examples of non-ribosomal peptides that are produced by one or more nonribosomal peptide synthetases are known in the art. One non-limiting example of non-ribosomal peptides that can be produced in accordance with the present invention is surfactin. Those of ordinary skill in the art will be aware of other non-ribosomal peptides that can be produced using compositions and methods of the present invention. In certain embodiments, a non-ribosomal peptide contains one or more covalently-linked fatty acid chains and is referred to herein as a lipopeptide (see definition of “lipopeptide”, supra).

“Polypeptide”: The term “polypeptide” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified. For example, a polypeptide may be glycosylated. Can comprise two or more polypeptides that function as a single active unit.

“Refined glycerol”: The term “refined glycerol” as used herein refers to glycerol is produced by subjecting crude glycerol (see definition of “crude glycerol”, supra) to art-recognized processes that remove contaminants and/or impurities. Refined glycerol is typically sold as a product that is at least 99.5% pure, although it will be recognized by those of ordinary skill in the art that the purity of refined glycerol may be lower that 99.5%. Processes to produce refined glycerol depend substantially on the type of impurities present in crude glycerol. For example, when crude glycerol is generated by hydrolysis, the starting crude glycerol is likely to be nearly 85% water, and multi-stage evaporators constructed of stainless steel are typically employed for concentration. Crude glycerol produced by other processes often has high salt content, and thin-film distillation is frequently employed. A summary containing some common purification processes is provided in Ullman's Encyclopedia of Chemical Technology, Vol. A-12, pages 480-483. As is discussed more fully herein, crude glycerol can also be produced as a byproduct of both biodiesel production and saponification. In both biodiesel production and saponification, the crude glycerol byproduct is subjected to one or more processes that remove contaminants and/or impurities to generate “refined glycerol”. As is known to those of ordinary skill in the art, such processes are laborious and time-consuming. “Crude glycerol” as the term is used herein refers to unprocessed or minimally processed glycerol that contains these and other contaminants and/or impurities. Removal of these contaminants and/or impurities results in what is defined herein as “refined glycerol”.

“Substantially lacks”: The term “substantially lacks” as used herein refers to the qualitative condition of exhibiting total or near-total absence of a particular component. One of ordinary skill in the biological arts will understand that biological and chemical compositions are rarely, if ever, 100% pure. Conversely, one of ordinary skill in the biological arts will understand that biological and chemical compositions are rarely, if ever, 100% free if a particular component. The term “substantially lacks” is therefore used herein to capture the concept that a biological and chemical composition may comprise a small, inconsequential amount of one or more impurities. To give but one particular example, when it is said that a cell culture medium “substantially lacks” a given component, it is meant to indicate that although a minute amount of that component may be present (for example, as a result of being an impurity and/or a breakdown product of one or more components of the cell culture medium), that component is nevertheless an inconsequential part of the cell culture medium and does not alter the basic properties of that cell culture medium. In certain embodiments, the term “substantially lacks”, as applied to a given component of a cell culture medium, refers to condition wherein the cell culture medium comprises less that 5%, 4%, 3%, 2%, 1%, 0.9%, 0.8%, 0.7%, 0.6%, 0.5%, 0.4%, 0.3%, 0.2%, 0.1% or less of that component. In certain embodiments, the term “substantially lacks”, as applied to a given component of a cell culture medium, refers to condition wherein the cell culture medium lacks any detectable amount of that component.

### Energy Sources

All living organisms require a carbon or energy source for growth, production of biologically useful molecules and metabolic activity generally. Microorganisms are known to utilize a wide variety of carbon sources, many of which are simple monosaccharide and disaccharide sugars such as, for example, glucose, dextrin, lactose, sucrose, maltose, fructose, and/or mannose. Additionally or alternatively, microorganisms are known to utilize a wide variety of non-sugar carbon sources such as, for example, starch and amino acids such as glutamate.

Although each of the carbon sources listed above are used to grow microorganisms, those of ordinary skill in the art do not employ each of these carbon sources to the same extent. For example, glucose is relatively abundant and cheap. Thus, glucose is a common carbon source for use in growing microorganisms. In addition to cost and availability, the choice of which carbon source to use in the culturing of microorganisms is determined by a variety of other factors including considerations such as the ability of the microorganism to utilize a particular carbon source, the ability of the microorganism to convert a particular carbon source into a product of interest, the type and amount of byproducts produced as a result of metabolizing the carbon source, etc. Clearly, having more options as to which carbon source to use will provide the practitioner more flexibility in choosing an appropriate and/or advantageous carbon source, depending on his or her practical, experimental, commercial and/or other needs.

Although glycerol is known to function as a carbon source for growth of microorganisms, crude glycerol was not traditionally considered to be a suitable carbon source. One of the primary reasons for this perceived unsuitability of crude glycerol stemmed from the presence of contaminants and/or impurities that were thought to be detrimental to growth of microorganisms. Indeed, several groups have shown that certain microorganisms cannot be grown in media containing crude glycerol. For example, Petitdemange et al. show that many strains of Clostridium butyricum did not grow in media containing industrial (crude) glycerol, including several widely used laboratory strains. See Petitdemange et al., Fermentation of raw glycerol to 1,3-propanediol by new strains of Clostridium butyricum, J. Indus. Micb., 15, 498-502, 1995). Furthermore, the majority of ten new Clostridium strains isolated from natural mud samples did not grow in media containing crude glycerol as a carbon source. Thus, even most naturally-occurring Clostridium species that are routinely exposed to toxins and other detrimental substances in their natural environment are adversely affected by the presence of contaminants and/or impurities present in crude glycerol. Papanikolaou et al. demonstrated that Yarrowia lipolytica can grow in media containing crude glycerol as a carbon source. See Papanikolaou et al., Yarrowia lipolytica as a potential producer of citric acid from raw glycerol, J. Appl. Micb., 92, 737-744, 2002). However, Papanikolaou et al. acknowledge that this result was unexpected, noting that many microorganisms are inhibited by contaminants and/or impurities present in crude glycerol. Thus, given the current knowledge in the art, it is not possible to predict from the outset whether a given microorganism will grow in media containing crude glycerol as a carbon source. In fact, to date the vast majority of microorganisms tested cannot grow in the presence of crude glycerol. The present disclosure demonstrates for the first time that laboratory strains of both the bacterium Bacillus subtilis and the yeast Saccharomyces cerevisiae are able to grow in culture media containing crude glycerol as the sole carbon source.

Given the perceived unsuitability of crude glycerol as a carbon source, practitioners that desired to utilize glycerol as a carbon source typically utilized refined glycerol. Refined glycerol is generated by subjecting crude glycerol to a variety of expensive and time-consuming purification steps to remove contaminants and/or impurities. Due to the cost and time required to generate refined glycerol, it is not a preferred carbon source for the growth of microorganisms, or for the production of compounds of interest such as polypeptides, peptides, lipopeptides and/or organic acids.

The present invention encompasses the recognition that certain microorganisms traditionally thought to be susceptible to the detrimental effects of contaminants and/or impurities present in crude glycerol can be advantageously grown in cell culture media containing crude glycerol as a carbon source. For example, the present invention demonstrates for the first time that both the bacteria Bacillus subtilis and the yeast Saccharomyces cerevisiae can be grown in cell culture media containing crude glycerol. In certain embodiments, microorganisms are grown in inventive cell culture media that contain crude glycerol as a carbon source, which inventive cell culture media further substantially lack refined glycerol. In certain embodiments, microorganisms are grown in inventive cell culture media that contain crude glycerol as the sole carbon source. In certain embodiments, microorganisms grown in inventive cell culture media that contain crude glycerol as a carbon source produce one or more compounds of interest. For example, such microorganisms may produce polypeptides, peptides, lipopeptides and/or organic acids, which can be isolated and optionally purified from the cell culture.

### Generation of Crude Glycerol

Crude glycerol is generated by a number of commercial and experimental processes, including saponification, e.g. hydrolysis of triacylglycerol to produce fatty acids and glycerol, and biodiesel production, in which transesterification of fats and oils occurs to produce methyl ester and a glycerol byproduct, and.

Biodiesel production involves the production of alkyl esters of long chain fatty acids by reacting the source acid with a low molecular weight alcohol, such as methanol. One of the byproducts of biodiesel production is crude glycerol. Since the introduction of biodiesel fuel in South Africa prior to World War II, work has proceeded to increase its viability as a fuel substitute. A traditional process for manufacturing fatty acid alkyl esters involves the transesterification of triglycerides using methanol, in the presence of an alkali catalyst. In addition to the desired fatty acid alkyl esters, this process produces an effluent stream comprising glycerol, excess alcohol, water, alkyl esters and a mixture of mono, di and triglycerides resulting from the transesterification step. The crude glycerol resulting from this process is likely to be combined with a wash water stream from the biodiesel purification, and that aqueous stream typically contains significant amount of methanol and sodium or potassium salts. Methanol is often removed by evaporation under vacuum conditions and utilizing falling film evaporators. Alternatively, methanol can be separated from the water and glycerin using a distillation column.

In more recent years, environmental and economic pressures (e.g., events such as oil embargoes, and laws such as the Clean Air Act of 1990) have provided impetus for continued development. Goals include production of biodiesel with cleaner burning properties and improved cold-temperature flow characteristics. However, most effort to date has been focused on waste minimization, by-product separation technology, and/or by-product utilization. Production of biodiesel fuel by a methyl-esterification process as applied to soy oil, produces an effluent stream with twenty (20%) percent crude glycerol content. This crude glycerol was typically considered an undesirable waste product to be disposed of. While industrial uses for crude glycerol have been pursued, such pursuits were not undertaken with widespread vigor due to the costly purification steps that typically had to be performed to produce even a low grade product of questionable value. Previously proposed uses for crude glycerol included mixing with animal manure to form a fertilizer and mixing with feed for animals (see e.g., Ahn et al., A Low Waste Process For The Production Of Biodiesel, Sep. Sci. & Tech., 30(7-9), 1995). Additionally, research has shown that potential exists for use of bacteriologically transformed crude glycerol to form products useful in plastics production (see e.g., Chowdhury et al., Vegetable Oils: From Table To Gas Tank, Chem. Eng. Feb., 1993). Perhaps the most promising and economically viable use for crude glycerol to date, however, is conversion into mono and di-fatty acid esters of crude glycerol.

Saponification is the hydrolysis of an ester under basic conditions to form an alcohol and the salt of a carboxylic acid. Saponification is commonly used to refer to the reaction of a metallic alkali (base) with a fat or oil to form soap. For example, a triglyceride comprising three fatty acid chains linked to a glycerol backbone can be exposed to sodium hydroxide (and/or other alkaline reagents known to those of ordinary skill in the art) to generate soap and glycerol. As in the biodiesel process described above, glycerol produced as a result of various commercial saponification reactions is generally contains a significant amount of contaminants and/or impurities and is generally considered to be an undesirable byproduct of the commercial saponification reaction.

Previously, crude glycerol's primary commercial value was in the generation of refined glycerol. In contrast to crude glycerol, refined glycerol contained little or no contaminants and/or impurities. However, the cost and labor involved in production of refined glycerol from crude glycerol diminished much of its commercial value. As more fully explained herein, the present invention encompasses the recognition that certain microorganisms traditionally thought to be susceptible to the detrimental effects of contaminants and/or impurities present in crude glycerol can be advantageously grown in cell culture media containing crude glycerol as a carbon source. As such, the present invention confers new and significant utility and value on crude glycerol generated by various commercial processes, since the expensive and laborious steps of removing such contaminants and/or impurities can be eliminated.

### Production of Polypeptide, Non-ribosomal Peptides and Lipopeptides in Microorganisms Using Crude Glycerol as Energy Source

In certain embodiments, a microorganism grown in compositions of the present invention and/or according to methods of the present invention produces one or more products of interest. For example, a microorganism may produce a polypeptide, non-ribosomal peptide and/or a lipopeptide. As one non-limiting example, a microorganism may produce the lipopeptide surfactin. Surfactin is cyclic lipopeptide that is naturally produced by certain bacteria, including the Gram-positive endospore-forming bacteria Bacillus subtilis. Surfactin is an amphiphilic molecule (having both hydrophobic and hydrophilic properties) and is thus soluble in both organic solvents and water. Surfactin exhibits exceptional surfactant properties, making it a commercially valuable molecule. Surfactin consists of a seven amino acid peptide loop, and a hydrophobic fatty acid chain (beta-hydroxy myristic acid) thirteen to fifteen carbons long. The fatty acid chain allows surfactin to penetrate cellular membranes The peptide loop is composed of the amino acids glutamic acid, leucine, D-leucine, valine, aspartic acid, D-leucine and leucine. Glutamic acid and aspartic acid residues at positions 1 and 5 respectively, constitute a minor polar domain. On the opposite side, valine residue at position 4 extends down facing the fatty acid chain, making up a major hydrophobic domain.

Surfactin is synthesized by the linear nonribosomal peptide synthetase, surfactin synthetase is synthesized by the three surfactin synthetase subunits SrfA-A, SrfA-B, and SrfA-C. Each of the enzymes SrfA-A and SrfA-B consist of three amino acid activating modules, while the monomodular subunit SrfA-C adds the last amino acid residue to the heptapeptide. Additionally the SrfA-C subunit includes the thioesterase domain (“TE domain”), which catalyzes the release of the product via a nucleophilic attack of the beta-hydroxy of the fatty acid on the carbonyl of the C-terminal Leu of the peptide, cyclizing the molecule via formation of an ester.

Due to its surfactant properties, surfactin also functions as an antibiotic. For example, surfactin is known to be effective as an anti-bacterial, anti-viral, anti-fungal, anti-mycoplasma and hemolytic compound. As an anti-bacterial compound, surfactin it is capable of penetrating the cell membranes of all types of bacteria, including both Gram-negative and Gram-positive bacteria, which differ in the composition of their membrane. Gram-positive bacteria have a thick peptidoglycan layer on the outside of their phospholipid bilayer. In contrast, Gram-negative bacteria have a thinner peptidoglycan layer on the outside of their phospholipid bilayer, and further contain an additional outer lipopolysaccharide membrane. Surfactin's surfactant activity permits it to create a permeable environment for the lipid bilayer and causes disruption that solubilizes the membrane of both types of bacteria. In order for surfactin to carry out minimal antibacterial effects, the minimum inhibitory concentration (MIC) is typically in the range of 12-50 μg/ml.

In addition to its antibacterial properties, surfactin also exhibits antiviral properties, and is known to disrupt enveloped viruses such as HIV and HSV. Surfactin not only disrupts the lipid envelope of viruses, but also their capsids through ion channel formations. Surfactin isoforms containing fatty acid chains with 14 or 15 carbon atoms exhibited improved viral inactivation, thought to be due to improved disruption of the viral envelope.

Those of ordinary skill in the art will be aware of other polypeptides, non-ribosomal peptides and/or a lipopeptides that are produced by any of a variety of microorganisms and will be able to select an appropriate microorganism to produce a polypeptide, non-ribosomal peptide and/or a lipopeptide of interest by growing such a microorganism in compositions of the present invention and/or in accordance with methods of the present invention.

In certain embodiments, a microorganism used to produce a polypeptide, non-ribosomal peptide and/or a lipopeptide of interest when grown in compositions of the present invention and/or in accordance with methods of the present invention is a bacterium. Non-limiting examples of bacteria that can be grown in accordance with the present invention include bacteria of the genera Bacillus, Clostridium, Enterobacter, Klebsiella, Micromonospora, Actinoplanes, Dactylosporangium, Streptomyces, Kitasatospora, Amycolatopsis, Saccharopolyspora, Saccharothrix and Actinosynnema. In certain embodiments, a microorganism used to produce a polypeptide, non-ribosomal peptide and/or a lipopeptide in accordance with the present invention is a bacterium of the genus Bacillus. In certain embodiments, a microorganism used to produce a polypeptide, non-ribosomal peptide and/or a lipopeptide in accordance with the present invention is a bacterium of the species Bacillus subtilis. Those of ordinary skill in the art will be aware of other bacteria that can produce polypeptides, non-ribosomal peptides and/or a lipopeptides when grown in compositions of the present invention and/or in accordance with methods of the present invention.

In certain embodiments, a composition of the present invention used to grow a microorganism that produces one or more polypeptides, non-ribosomal peptides and/or a lipopeptides of interest comprises a complex cell culture medium. As recognized in the art, complex media typically contain at least one component whose identity or quantity is either unknown or uncontrolled. Non-limiting examples of components that may be added to complex media include yeast extract, bacto-peptone, and/or other hydrolysates. In certain embodiments, a microorganism grown in a complex medium of the present invention comprising crude glycerol as a carbon source produces a polypeptide, non-ribosomal peptide and/or a lipopeptide of interest in an amount that is nearly the amount of organic acid that would be produced if the microorganism were grown under otherwise identical conditions in a traditional complex medium. For example, a polypeptide, non-ribosomal peptide and/or a lipopeptide produced by a microorganism in accordance with the present invention may be produced in an amount that is at least 20%, 25%, 30%, 35%, 40%, 45%, 50%, 55%, 60%, 65%, 70%, 75%, 80%, 85%, 90%, 95%, 96%, 97%, 98%, 99% or more the amount of polypeptide, non-ribosomal peptide and/or a lipopeptide that would be produced if the microorganism were grown under otherwise identical conditions in a traditional complex medium. In certain embodiments, a microorganism grown in a complex medium of the present invention comprising crude glycerol as a carbon source produces a polypeptide, non-ribosomal peptide and/or a lipopeptide of interest in an amount that is equivalent to the amount of organic acid that would be produced if the microorganism were grown under otherwise identical conditions in a traditional complex medium. In certain embodiments, a microorganism grown in a complex medium of the present invention comprising crude glycerol as a carbon source produces a polypeptide, non-ribosomal peptide and/or a lipopeptide of interest in an amount that is greater than the amount of polypeptide, non-ribosomal peptide and/or a lipopeptide that would be produced if the microorganism were grown under otherwise identical conditions in a complex defined medium.

In certain embodiments, a composition of the present invention used to grow a microorganism that produces one or more polypeptides, non-ribosomal peptides and/or a lipopeptides of interest comprises a defined cell culture medium. A variety of chemically defined growth media for use in cell culture are known to those of ordinary skill in the art. Since each component of a defined medium is typically well characterized and present in known amounts, defined media do not contain complex additives such as serum or hydrolysates. Such defined media can be modified according to the teachings of the present disclosure to generate a cell culture medium that comprises crude glycerol as a carbon source. In certain embodiments, a defined medium of the present invention comprises crude glycerol as a carbon source, and further substantially lacks refined glycerol as an energy source. In certain embodiments, a defined medium of the present invention comprises crude glycerol as the sole carbon source.

In certain embodiments, a defined cell culture medium of the present invention comprises a limiting amount of one or more components. As one non-limiting embodiment, a cell culture medium of the present invention may comprise a limiting amount nitrogen. In certain embodiments, a microorganism grown in a defined medium of the present invention comprising crude glycerol as a carbon source, and further comprising a limiting amount of one or more components (e.g. nitrogen), produces a polypeptide, non-ribosomal peptide and/or a lipopeptide of interest in an amount that is nearly the amount of polypeptide, non-ribosomal peptide and/or a lipopeptide that would be produced if the microorganism were grown under otherwise identical conditions in a traditional defined medium. For example, a polypeptide, non-ribosomal peptide and/or a lipopeptide produced by a microorganism in accordance with the present invention may be produced in an amount that is at least 20%, 25%, 30%, 35%, 40%, 45%, 50%, 55%, 60%, 65%, 70%, 75%, 80%, 85%, 90%, 95%, 96%, 97%, 98%, 99% or more the amount of polypeptide, non-ribosomal peptide and/or a lipopeptide that would be produced if the microorganism were grown under otherwise identical conditions in a traditional defined medium. In certain embodiments, a microorganism grown in a defined medium of the present invention comprising crude glycerol as a carbon source, and further comprising a limiting amount of one or more components (e.g. nitrogen), produces a polypeptide, non-ribosomal peptide and/or a lipopeptide of interest in an amount that is equivalent to the amount of polypeptide, non-ribosomal peptide and/or a lipopeptide that would be produced if the microorganism were grown under otherwise identical conditions in a traditional defined medium. In certain embodiments, a microorganism grown in a defined medium of the present invention comprising crude glycerol as a carbon source, and further comprising a limiting amount of one or more components (e.g. nitrogen), produces a polypeptide, non-ribosomal peptide and/or a lipopeptide of interest in an amount that is greater than the amount of organic acid that would be produced if the microorganism were grown under otherwise identical conditions in a traditional defined medium.

In certain embodiments, a microorganism grown in a defined or complex medium of the present invention comprising crude glycerol as a carbon source produces a polypeptide, non-ribosomal peptide and/or a lipopeptide to a level of 0.1 g/L, 0.2 g/L, 0.3 g/L, 0.4 g/L, 0.5 g/L, 0.6 g/L, 0.7 g/L, 0.8 g/L, 0.9 g/L, 1.0 g/L, 2.0 g/L, 3.0 g/L, 4.0 g/L, 5.0 g/L, 6.0 g/L, 7.0 g/L, 8.0 g/L, 9.0 g/L, 10 g/L, 20 g/L, 30 g/L, 40 g/L, 50 g/L, 60 g/L, 70 g/L, 80 g/L, 90 g/L, 100 g/L, or more.

In certain embodiments, the amount of polypeptide, non-ribosomal peptide and/or lipopeptide of interest produced is increased by subjecting a cell culture containing a microorganism that produces the polypeptide, non-ribosomal peptide and/or lipopeptide to one or more methods of the present invention. In certain embodiments, the production of a polypeptide, non-ribosomal peptide and/or lipopeptide of interest is supplementing the cell culture with a nitrogen source such as without limitation, tryptone, total soy extract, yeast extract, casamino acids and/or distiller grains. In certain embodiments, a microorganism produces a polypeptide, non-ribosomal peptide, and/or lipopeptide of interest to an increased level relative to the level of polypeptide, non-ribosomal peptide, and/or lipopeptide that would be produced by a microorganism grown under otherwise identical conditions in an otherwise identical cell culture medium that lacks the provided nitrogen source. In certain embodiments, a nitrogen source added to the cell culture increases production of the polypeptide, non-ribosomal peptide and/or lipopeptide of interest by a relatively greater amount than amount by which the total biomass of the cell culture is increased. In such embodiments, a polypeptide, non-ribosomal peptide, and/or lipopeptide of interest produced in a cell culture to which the nitrogen source is added represents an increased fraction of the total biomass of the cell culture compared the fraction that would result if the nitrogen source were not added to the cell culture.

As is known to those of ordinary skill in the art, cell cultures typically undergo a growth phase, in which rapid cell doubling takes place. During the growth phase, the number of cells in the culture is low enough such that nutrients, energy sources and other culture components are not limiting. As a result, the cells have an ample supply of such nutrients, energy sources and other culture components. After the growth phase, a cell culture typically reaches what is known as stationary phase. During stationary phase, the number of cells in the culture has increased to a point where nutrients, energy sources and other culture components become limiting. As a result, the rate of cell doubling declines or stops, and cell density may even decline. Cell density of a cell culture can be determined in any of a variety of ways. For example, cell density can be determined by measuring the optical density (“OD”) of the cell culture. OD650, defined as the optical density at a wavelength of 650 nm, is a useful and recognized wavelength at which to measure cell density.

In certain embodiments, a nitrogen source such as tryptone, total soy extract, yeast extract, casamino acids and/or distiller grains is added after the cell culture has reached stationary phase. For example, a nitrogen source may be added once the OD650 of a cell culture stops increasing, or begins to decline (e.g., by 5%, 10%, or any other amount recognized by those skilled in the art as being indicative of stationary phase). Those of ordinary skill in the art will be aware of other ways to determine whether a cell culture has reached stationary phase. In certain embodiments, a cell culture is grown to stationary phase at a defined temperature or temperature range prior to nitrogen addition. For example, a cell culture may be grown to stationary phase at a temperature of about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., or a temperature range encompassing two of these temperatures. In certain embodiments, a cell culture is grown to stationary phase at about 30° C. In certain embodiments, a cell culture is grown to stationary phase at about 37° C. In certain embodiments, a cell culture is grown to stationary phase at about 45° C. Those of ordinary skill in the art will be able to choose an appropriate and/or advantageous temperature or temperature range at which to grow cells to stationary phase based on any of a variety of considerations including, but not limited to, the nature of the cell and/or the nature of the produced polypeptide, non-ribosomal peptide and/or lipopeptide.

In certain embodiments, a nitrogen source is added after the cell culture has reached stationary phase due to the fact that addition of a nitrogen source prior to this stage could contribute to in an increase in cell density without a concomitant increase in the amount of polypeptide, non-ribosomal peptide and/or lipopeptide produced. In certain embodiments, adding such a nitrogen source during the growth phase may contribute to a decrease in the amount of polypeptide, non-ribosomal peptide and/or lipopeptide produced. As will be recognized by those of ordinary skill in the art, such decreases in production are preferably avoided or minimized.

In certain embodiments, a decrease in the amount of polypeptide, non-ribosomal peptide and/or lipopeptide produced can occur when a nitrogen source is added during growth phase of a cell culture grown at a relatively low temperature or temperature range. A “relatively low temperature or temperature range”, as the phrase is used here, refers to a temperature of about 25° C., about 26° C., about 27° C., about 28° C., about 29° C., about 30° C., about 31° C., about 32° C., about 33° C., about 34° C., about 35° C., about 36° C., or a temperature range encompassing two of these temperatures. In certain embodiments, a decrease in the amount of polypeptide, non-ribosomal peptide and/or lipopeptide produced can occur when a nitrogen source is added during growth phase of a cell culture grown at a relatively low temperature of about 30° C.

In certain embodiments, a decrease in the amount of polypeptide, non-ribosomal peptide and/or lipopeptide produced is prevented or minimized by adding the nitrogen source once a cell culture grown at a relatively low temperature or temperature range has reached stationary phase instead of adding the nitrogen source during the growth phase. For example, the present inventors have discovered that when grown at the relatively low temperature of 30° C., addition of tryptone and/or yeast extract in growth phase to a culture of Bacillus subtilis producing surfactin may contribute to an increase in cell density and a decrease in the amount of surfactin produced. However, when such cells are grown at the same relatively low temperature of 30° C. under otherwise identical conditions, adding tryptone and/or yeast extract after the culture has reached stationary phase results in both an increase in production of surfactin and an increase in cell density. Thus, the present invention encompasses the broader recognition that, in certain embodiments, growing cells to stationary phase at a relatively low temperature prior to adding a nitrogen source results in an increase in the amount of a produced polypeptide, non-ribosomal peptide and/or lipopeptide produced by those cells.

In certain embodiments, a nitrogen source such as tryptone, total soy extract, yeast extract, casamino acids and/or distiller grains are added while the cell culture is in growth phase. For example, the present inventors have discovered that whether a decrease in surfactin production occurs after adding a nitrogen source during growth phase of a cell culture grown at a relatively low temperature or temperature range can depend on the nature of the nitrogen source itself. As shown in Example 7, distillers grains added during growth phase to a cell culture grown at the relatively low temperature of 30° C., which cell culture comprises a population of Bacillus subtilis that produces surfactin, results in an increase in surfactin production, while addition of tryptone and/or yeast extract to a similar culture grown at the relatively low temperature of 30° C. under otherwise identical conditions may contribute to a decrease in surfactin production. Thus, in certain embodiments, production of a polypeptide, non-ribosomal peptide and/or lipopeptide is increased by supplementing a cell culture with distillers grains during growth phase. As described more fully below, an increase in production of the lipopeptide surfactin can also be achieved if a cell culture is supplemented with distillers grain as a nitrogen source, wherein the cell culture is grown at a relatively high temperature or temperature range. A “relatively high temperature or temperature range”, as the phrase is used herein, refers to a temperature of about 37° C., about 38° C., about 39° C., about 40° C., about 41° C., about 42° C., about 43° C., about 44° C., about 45° C., about 46° C., about 47° C., about 48° C., about 49° C., about 50° C., or a temperature range encompassing two of these temperatures. In certain embodiments, a cell culture is grown at a relatively high temperature of about 37° C. In certain embodiments, a cell culture is grown at a relatively high temperature of about 45° C.

In certain embodiments, a nitrogen source such as tryptone, total soy extract, yeast extract, casamino acids and/or distiller grains are added while the cell culture is in growth phase, which culture is grown at a relatively high temperature or temperature range. For example, as shown in Example 7, addition of a nitrogen source during growth phase of a cell culture grown at the relatively high temperature of either 37° C. or 45° C. results in an increase in the amount of surfactin produced by a Bacillus subtilis culture. The increase in surfactin production is accompanied by a concomitant increase in cell density.

Thus, the present disclosure demonstrates that production of a polypeptide, non-ribosomal peptide and/or lipopeptide of interest may be increased by supplementing a cell culture with distillers grains during growth phase when the cell culture is grown at a relatively low temperature or temperature range (e.g., 30° C.). The present disclosure further demonstrates that production of a polypeptide, non-ribosomal peptide and/or lipopeptide of interest may be increased by supplementing a cell culture with a nitrogen source such as, without limitation, tryptone, total soy extract, yeast extract, casamino acids and/or distiller grains, during growth phase when the cell culture is grown at a relatively high temperature or temperature range (e.g., 37° C. or 45° C.).

In certain embodiments, the yield of a polypeptide, non-ribosomal peptide and/or a lipopeptide of interest produced by a microorganism grown in inventive media containing crude glycerol is at least about 10%, 15%, 20%, 25%, 30%, 35%, 40%, 45%, 50%, 55%, 56%, 57%, 58%, 59%, 60%, 65%, 70%, 75%, 80%, 85%, 90% or more. Yield is defined as the amount of carbon source (e.g., crude glycerol) that is converted to product (e.g., a polypeptide, non-ribosomal peptide and/or a lipopeptide). Thus, if 50% of crude glycerol is converted to a polypeptide, non-ribosomal peptide and/or a lipopeptide, the yield is 50%. The theoretical maximal yield of surfactin produced from crude glycerol is calculated to be 60%. As disclosed herein (see e.g., Example 3 and Example 4), the present inventors have discovered that yields approaching this theoretical maximal yield can be achieved by growing Bacillus subtilis in inventive cell culture media containing crude glycerol as the sole carbon source. Given that crude glycerol has traditionally been considered in inferior carbon source for growth of microorganisms, the exhibited high crude glycerol/surfactin yield represents a significant advance in the commercial utility of using crude glycerol as a carbon source for the production of commercially valuable products such as surfactin.

In certain embodiments, a microorganism grown in a defined medium of the present invention comprising crude glycerol as a carbon source grows to a cell density that is comparable to the cell density that would be achieved if the microorganism were grown under otherwise identical conditions in a traditional defined medium. For example, a microorganism grown in a defined medium of the present invention comprising crude glycerol as a carbon source may grow to a cell density that is at least 50%, 55%, 60%, 65%, 70%, 75%, 80%, 85%, 90%, 95%, 96%, 97%, 98%, 99% or greater than the cell density that would be achieved if the microorganism were grown under otherwise identical conditions in a traditional defined medium. In certain embodiments, a microorganism grown in a defined medium of the present invention comprising crude glycerol as a carbon source grows to a cell density that is greater than the cell density that would be achieved if the microorganism were grown under otherwise identical conditions in a traditional defined medium. For example, a microorganism grown in a defined medium of the present invention comprising crude glycerol as a carbon source may grow to a cell density that is at least 100%, 110%, 120%, 130%, 140%, 150% or greater than the cell density that would be achieved if the microorganism were grown under otherwise identical conditions in a traditional defined medium.

### Production of Organic Acids in Microorganisms Using Crude Glycerol as Energy Source

In certain embodiments, a microorganism grown in compositions of the present invention and/or according to methods of the present invention produces one or more products of interest. For example, a microorganism may produce an organic acid. As non-limiting examples, a microorganism may produce an organic acid selected from the group consisting of pyruvic acid, succinic acid, fumaric acid, malic acid, maleic acid, citric acid and combinations thereof. Those of ordinary skill in the art will be aware of other organic acids that are produced by any of a variety of microorganisms and will be able to select an appropriate microorganism to produce an organic acid of interest by growing such a microorganism in compositions of the present invention and/or in accordance with methods of the present invention.

In certain embodiments, a microorganism used to produce an organic acid of interest when grown in compositions of the present invention and/or in accordance with methods of the present invention is a fungus. Non-limiting examples of fungi that can be grown in accordance with the present invention include yeast of the genera Saccharomyces, Pichia, Aspergillus, Trichoderma, Kluyveromyces, Candida, Hansenula, Schizpsaccaromyces, Yarrowia, and Chrysoporium. Those of ordinary skill in the art will be aware of other fungi that can produce organic acids when grown in compositions of the present invention and/or in accordance with methods of the present invention. In certain embodiments, a microorganism used to produce an organic acid in accordance with the present invention is a yeast of the genus Saccharomyces. In certain embodiments, a microorganism used to produce an organic acid in accordance with the present invention is a yeast of the species Saccharomyces cerevisiae.

Saccharomyces cerevisiae is among the first cellular organisms utilized by humans and continues to serve as a model eukaryotic organism for biological research. The extensive level of biochemical characterization of Saccharomyces cerevisiae metabolism achieved to date is a result of a thorough understanding of growth and fermentation conditions as well as the ease with which this yeast organism can be genetically manipulated. These factors combine to make this yeast organism an ideal platform for bioengineering efforts.

Growth of Saccharomyces cerevisiae requires the presence of a carbon source to support metabolic functions. Dextrose (glucose) is the preferred carbon source under aerobic conditions as an overwhelming body of evidence supports the production of metabolites to high concentrations with its use (Barnett, J. A., Payne, R. W., and Yarrow, D., Yeasts: characteristics and identification, 1st Ed., Cambridge University Press, Cambridge, 1983). However, S. cerevisiae is capable of using a variety of fermentable and non-fermentable sugars as carbon sources, increasing the versatility of this organism as an industrial platform for chemical production (see for example, Grannot and Snyder, Carbon source induces growth of stationary phase yeast cells, independent of carbon source metabolism, Yeast, May; 9(5):465-79, 1993).

Methods and compositions of the present invention expand the utility of Saccharomyces cerevisiae and other microorganisms as industrial platforms for chemical production. For example, due to the detrimental effects of contaminants and/or impurities present in crude glycerol, cell culture media containing crude glycerol were traditionally thought to be unsuitable for growth of microorganisms and/or production of compounds of interest (including for example, but not limited to, organic acids). The present invention demonstrates that Saccharomyces cerevisiae and other microorganisms can be grown in cell culture media containing crude glycerol as a carbon source, and that such organisms can produce commercially important products such as, for example, organic acids.

In certain embodiments, Saccharomyces cerevisiae is grown in a cell culture medium comprising crude glycerol as a carbon source. In certain embodiments, Saccharomyces cerevisiae is grown in a cell culture medium that comprises crude glycerol as an energy source, which cell culture medium further substantially lacks refined glycerol. In certain embodiments, Saccharomyces cerevisiae is grown in a cell culture medium that comprises crude glycerol as the sole energy source. In certain embodiments, Saccharomyces cerevisiae grown in such cell culture media produce an organic acid selected from the group consisting of pyruvic acid, succinic acid, fumaric acid, malic acid, maleic acid, citric acid and combinations thereof.

In certain embodiments, a microorganism used to produce an organic acid of interest when grown in compositions of the present invention and/or in accordance with methods of the present invention is a bacterium. Non-limiting examples of bacteria that can be grown in accordance with the present invention include bacteria of the genera Bacillus, Clostridium, Enterobacter, Klebsiella, Micromonospora, Actinoplanes, Dactylosporangium, Streptomyces, Kitasatospora, Amycolatopsis, Saccharopolyspora, Saccharothrix and Actinosynnema. In certain embodiments, a microorganism used to produce an organic acid in accordance with the present invention is a bacterium of the genus Clostridium. In certain embodiments, a microorganism used to produce an organic acid in accordance with the present invention is a bacterium of the species Clostridium butyricum. In certain embodiments, a microorganism used to produce an organic acid in accordance with the present invention is a bacterium of the genus Enterobacter. In certain embodiments, a microorganism used to produce an organic acid in accordance with the present invention is a bacterium of the species Enterobacter aerogenes. In certain embodiments, a microorganism used to produce an organic acid in accordance with the present invention is a bacterium of the genus Klebsiella. In certain embodiments, a microorganism used to produce an organic acid in accordance with the present invention is a bacterium of the species Klebsiella pneumoniae. Those of ordinary skill in the art will be aware of other bacteria that can produce organic acids when grown in compositions of the present invention and/or in accordance with methods of the present invention.

In certain embodiments, a composition of the present invention used to grow a microorganism that produces one or more organic acids of interest comprises a complex cell culture medium. As recognized in the art, complex media typically contain at least one component whose identity or quantity is either unknown or uncontrolled. Non-limiting examples of components that may be added to complex media include yeast extract, bacto-peptone, and/or other hydrolysates. In certain embodiments, a microorganism grown in a complex medium of the present invention comprising crude glycerol as a carbon source produces an organic acid of interest in an amount that is nearly the amount of organic acid that would be produced if the microorganism were grown under otherwise identical conditions in a traditional complex medium. For example, an organic acid produced by a microorganism in accordance with the present invention may be produced in an amount that is at least 20%, 25%, 30%, 35%, 40%, 45%, 50%, 55%, 60%, 65%, 70%, 75%, 80%, 85%, 90%, 95%, 96%, 97%, 98%, 99% or more the amount of organic acid that would be produced if the microorganism were grown under otherwise identical conditions in a traditional complex medium. In certain embodiments, a microorganism grown in a complex medium of the present invention comprising crude glycerol as a carbon source produces an organic acid of interest in an amount that is equivalent to the amount of organic acid that would be produced if the microorganism were grown under otherwise identical conditions in a traditional complex medium. In certain embodiments, a microorganism grown in a complex medium of the present invention comprising crude glycerol as a carbon source produces an organic acid of interest in an amount that is greater than the amount of organic acid that would be produced if the microorganism were grown under otherwise identical conditions in a complex defined medium.

In certain embodiments, a composition of the present invention used to grow a microorganism that produces one or more organic acids of interest comprises a defined cell culture medium. A variety of chemically defined growth media for use in cell culture are known to those of ordinary skill in the art. Since each component of a defined medium is typically well characterized and present in known amounts, defined media do not contain complex additives such as serum or hydrolysates. Such defined media can be modified according to the teachings of the present disclosure to generate a cell culture medium that comprises crude glycerol as a carbon source. In certain embodiments, a defined medium of the present invention comprises crude glycerol as a carbon source, and further substantially lacks refined glycerol as an energy source. In certain embodiments, a defined medium of the present invention comprises crude glycerol as the sole carbon source.

In certain embodiments, a defined cell culture medium of the present invention comprises a limiting amount of one or more components. As one non-limiting embodiment, a cell culture medium of the present invention may comprise a limiting amount nitrogen. In certain embodiments, a microorganism grown in a defined medium of the present invention comprising crude glycerol as a carbon source, and further comprising a limiting amount of one or more components (e.g. nitrogen), produces an organic acid of interest in an amount that is nearly the amount of organic acid that would be produced if the microorganism were grown under otherwise identical conditions in a traditional defined medium. For example, an organic acid produced by a microorganism in accordance with the present invention may be produced in an amount that is at least 20%, 25%, 30%, 35%, 40%, 45%, 50%, 55%, 60%, 65%, 70%, 75%, 80%, 85%, 90%, 95%, 96%, 97%, 98%, 99% or more the amount of organic acid that would be produced if the microorganism were grown under otherwise identical conditions in a traditional defined medium. In certain embodiments, a microorganism grown in a defined medium of the present invention comprising crude glycerol as a carbon source, and further comprising a limiting amount of one or more components (e.g. nitrogen), produces an organic acid of interest in an amount that is equivalent to the amount of organic acid that would be produced if the microorganism were grown under otherwise identical conditions in a traditional defined medium. In certain embodiments, a microorganism grown in a defined medium of the present invention comprising crude glycerol as a carbon source, and further comprising a limiting amount of one or more components (e.g. nitrogen), produces an organic acid of interest in an amount that is greater than the amount of organic acid that would be produced if the microorganism were grown under otherwise identical conditions in a traditional defined medium.

In certain embodiments, a microorganism grown in a defined or complex medium of the present invention comprising crude glycerol as a carbon source produces an organic acid to a level of 0.2 μg/mL, 0.3 μg/mL, 0.4 μg/mL, 0.5 μg/mL, 0.6 μg/mL, 0.7 μg/mL, 0.8 μg/mL, 0.9 μg/mL, 1.0 μg/mL, 2.0 μg/mL, 3.0 μg/mL, 4.0 μg/mL, 5.0 μg/mL, 6.0 μg/mL, 7.0 μg/mL, 8.0 μg/mL, 9.0 μg/mL, 10 μg/mL, 20 μg/mL, 30 μg/mL, 40 μg/mL, 50 μg/mL, 60 μg/mL, 70 μg/mL, 80 μg/mL, 90 μg/mL, 100 μg/mL, 125 μg/mL, 150 μg/mL, 175 μg/mL, 200 μg/mL, 300 μg/mL, 400 μg/mL, 500 μg/mL, 600 μg/mL, 700 μg/mL, 800 μg/mL, 900 μg/mL, 1.0 mg/mL, 2.0 mg/mL, 3.0 mg/mL, 4.0 mg/mL, 5.0 mg/mL, 6.0 mg/mL, 7.0 mg/mL, 8.0 mg/mL, 9.0 mg/mL, 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 125 mg/mL, 135 mg/mL, 150 mg/mL, 175 mg/mL, 200 mg/mL, 300 mg/mL, 400 mg/mL, 500 mg/mL, or more.

In certain embodiments, the yield of an organic acid of interest produced by a microorganism grown in inventive media containing crude glycerol is at least about 10%, 15%, 20%, 25%, 30%, 35%, 40%, 45%, 50%, 55%, 56%, 57%, 58%, 59%, 60%, 65%, 70%, 75%, 80%, 85%, 90% or more. Yield is defined as the amount of carbon source (e.g., crude glycerol) that is converted to product (e.g., an organic acid). Thus, if 50% of crude glycerol is converted to an organic acid, the yield is 50%.

In certain embodiments, the relative production ratio of two or more organic acids produced by a microorganism is altered as a result of growing such a microorganism in cell culture media of the present invention and/or according to methods of the present invention. For example, the production of a first organic acid may be increased at the expense of production of a second organic acid. Additionally or alternatively, when grown in cell culture media of the present invention and/or according to methods of the present invention, but the production of a first organic acid is increased to a greater extent than the production of a second organic acid. Additionally or alternatively, the production of each of two organic acids may be decreased when grown in cell culture media of the present invention and/or according to methods of the present invention, but the production of a first organic acid is decreased to a lesser extent than the production of a second organic acid. For example, as shown in Example 5, the level of pyruvic acid produced by Saccharomyces cerevisiae grown in cell culture media comprising crude glycerol as a carbon source were reduced by 94% compared to the amount of pyruvic acid produced by Saccharomyces cerevisiae grown in cell culture media comprising glucose as a carbon source, whereas the level of succinic acid produced was only reduced by 54%. Other organic acids produced by Saccharomyces cerevisiae grown in cell culture media comprising glucose as a carbon source in Example 5 exhibited a reduction between that of pyruvic acid and succinic acid.

In certain embodiments, a microorganism grown in a defined medium of the present invention comprising crude glycerol as a carbon source grows to a cell density that is equivalent to the cell density that would be achieved if the microorganism were grown under otherwise identical conditions in a traditional defined medium. As shown in Example 5, a comparable cell density can be achieved when such a microorganism is grown either in complex media or in defined media, including defined media comprising a limiting amount of one or more components (e.g. nitrogen).

In certain embodiments, a microorganism grown in a defined medium of the present invention comprising crude glycerol as a carbon source grows to a cell density that comparable to the cell density that would be achieved if the microorganism were grown under otherwise identical conditions in a traditional defined medium. For example, a microorganism grown in a defined medium of the present invention comprising crude glycerol as a carbon source may grow to a cell density that is at least 50%, 55%, 60%, 65%, 70%, 75%, 80%, 85%, 90%, 95%, 96%, 97%, 98%, 99% or greater than the cell density that would be achieved if the microorganism were grown under otherwise identical conditions in a traditional defined medium. In certain embodiments, a microorganism grown in a defined medium of the present invention comprising crude glycerol as a carbon source grows to a cell density that is greater than the cell density that would be achieved if the microorganism were grown under otherwise identical conditions in a traditional defined medium. For example, a microorganism grown in a defined medium of the present invention comprising crude glycerol as a carbon source may grow to a cell density that is at least 100%, 110%, 120%, 130%, 140%, 150% or greater than the cell density that would be achieved if the microorganism were grown under otherwise identical conditions in a traditional defined medium.

### EXAMPLES

### Example 1

### Determining Advantageous Concentration of Soy Extract

In order to minimize the cost of raw materials needed to grow Bacillus subtilis cells capable of producing surfactin and surfactin-analogs, we decided to investigate the use of low cost feed media containing soy extract and crude glycerol (˜80%) as a carbon source. Soy and crude glycerol are waste byproducts of soy oil recovery and chemical processes, respectively. In an initial experiment, we tested the concentration of soy extract (Sigma) that results in the highest cell density using 0.5% pure glycerol (American Bioanalytical) as a carbon source. The test media also contained minimal salts (6 g Na₂HPO₄, 3 g KH₂PO₄, 0.5 g NaCl, 1 g NH₄Cl, per liter).

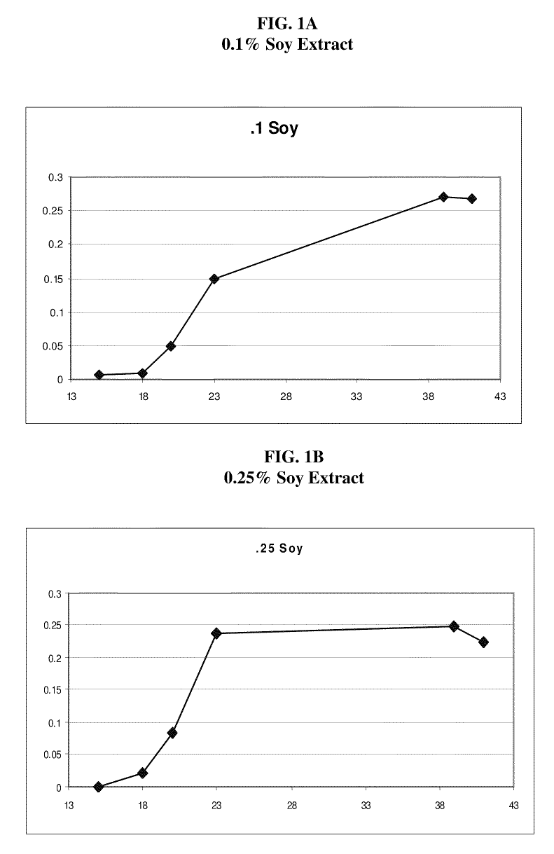

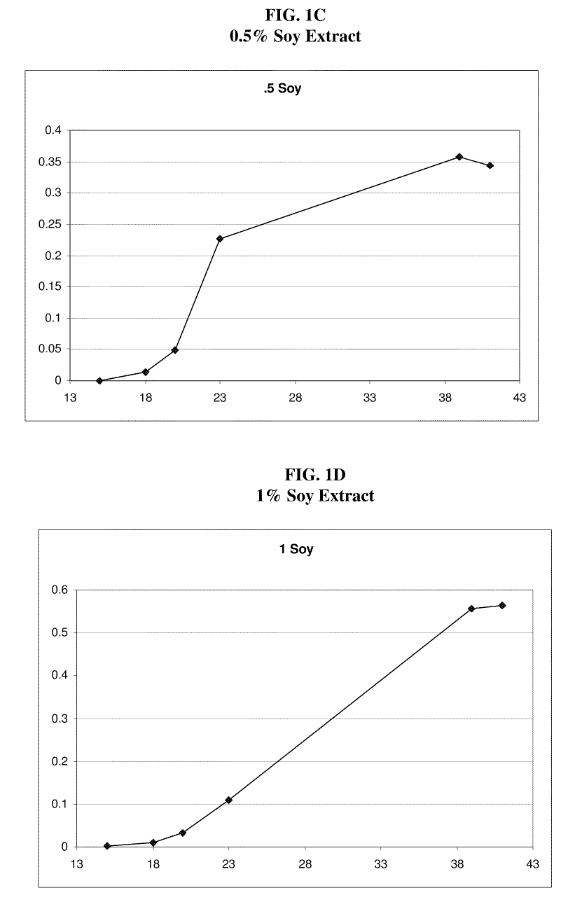

The growth curves of the cultures grown in various concentrations of soy extract are shown in FIG. 1.

### Example 2

### Growth of Bacillus subtilis on Crude Glycerol

Following the results obtained in Example 1, we tested the ability of Bacillus subtilis (strain OKB105 Δ(upp)Spect^{R}) to grow in crude glycerol using 1% soy extract. Crude glycerol used in this example was obtained from World Energy, Inc., 1248 George Jenkins Blvd Suite J-4, Lakeland, Fla., 33815 and was derived from soy bean oil using a process designed to produce biodiesel. To test the quality of the ˜80% crude glycerol, we carried out control experiments using pure glycerol. To make crude glycerol suitable for bacterial growth, crude glycerol was autoclaved and then filtered through a 0.2 μm filter.

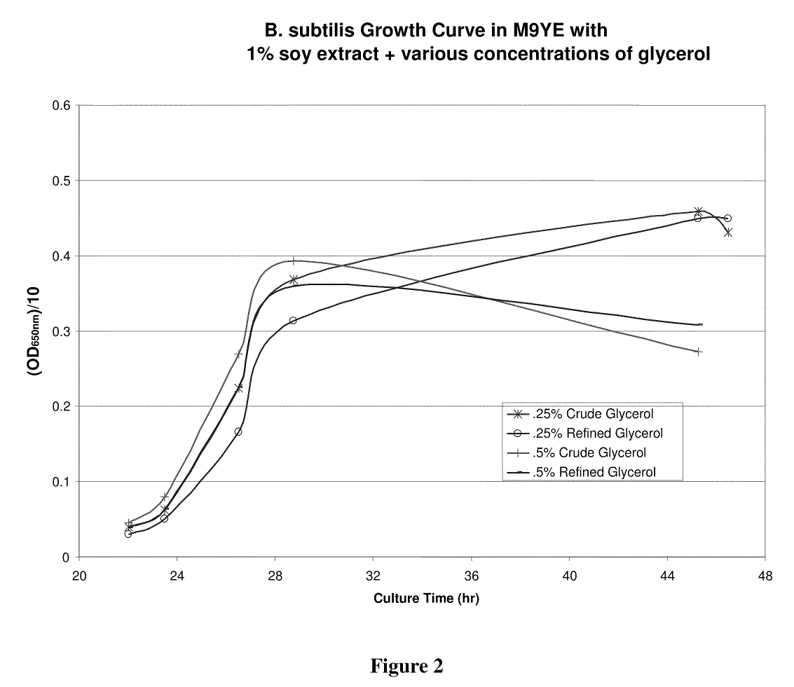

These media for these experiments consisted of minimal salts as described above, 1% soy extract, and 0.25% or 0.5% pure and crude glycerol, separately. In all cases, after mixing media components, insoluble material present in the media was removed during filter-sterilization. The growth curves of strain OKB105 Δ(upp)Spect^{R} are shown in FIG. 2. As can be seen, the growth curves of cell cultures using crude glycerol as a carbon source are substantially the same as cultures using refined glycerol as a carbon source.

Following this experiment, we tested the ability of this strain to produce surfactin using pure and crude glycerol at 0.25% and 0.5%, two concentrations of soy extract (0.25% and 1%), and each of the growth conditions was supplemented with both 2% and 4% of soy extract, separately. Supplementation occurred when cells reached saturation, as described below. All growth conditions are summarized in Table 1.

TABLE 1
Glycerol and Soy Extract Conditions

| | Pure | Crude | Soy extract | Soy extract (%) |
|---|---|---|---|---|
| Condition | Glycerol (%) | Glycerol (%) | (%) | supplementation |
| 1 | 0.25 | 0 | 0.25 | 2 |
| 2 | 0 | 0.25 | 0.25 | 2 |
| 3 | 0.25 | 0 | 1 | 2 |
| 4 | 0 | 0.25 | 1 | 2 |
| 5 | 0.5 | 0 | 0.25 | 2 |
| 6 | 0 | 0.5 | 0.25 | 2 |
| 7 | 0.5 | 0 | 1 | 2 |
| 8 | 0 | 0.5 | 1 | 2 |
| 9 | 0.25 | 0 | 0.25 | 4 |
| 10 | 0 | 0.25 | 0.25 | 4 |
| 11 | 0.25 | 0 | 1 | 4 |
| 12 | 0 | 0.25 | 1 | 4 |
| 13 | 0.5 | 0 | 0.25 | 4 |
| 14 | 0 | 0.5 | 0.25 | 4 |
| 15 | 0.5 | 0 | 1 | 4 |
| 16 | 0 | 0.5 | 1 | 4 |

All cultures were grown overnight at 30° C. When the absorbance at 650 nm began to drop, cells were supplemented with either 2 mL or 4 mL of 10% soy extract. Supplementation occurred at 29 hours after inoculation for the 0.25% soy extract/0.25% glycerol and at ˜46-47 hours for the remaining combinations. As a control, cells were also grown in M9YE with 0.5% glucose as a carbon source. Approximately 24 hrs after inoculation, control cells were supplemented with 40 g/l of tryptone.

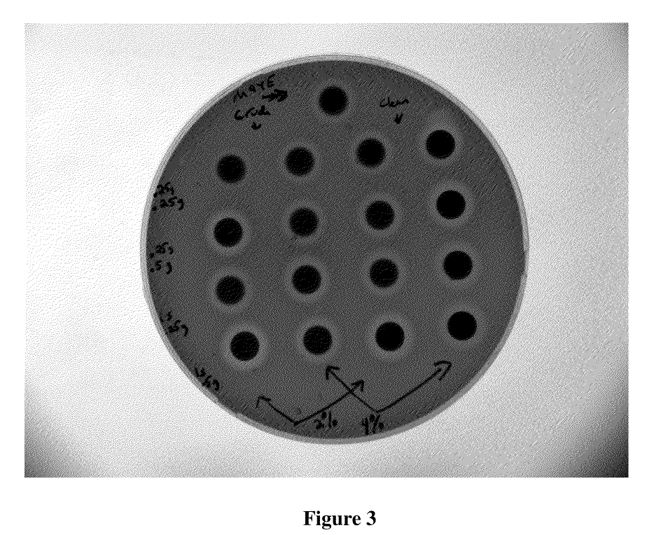

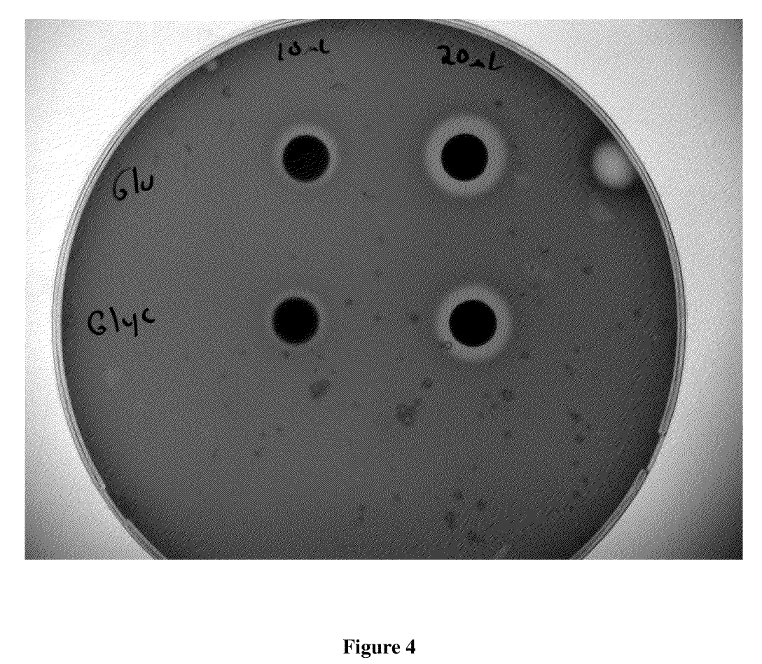

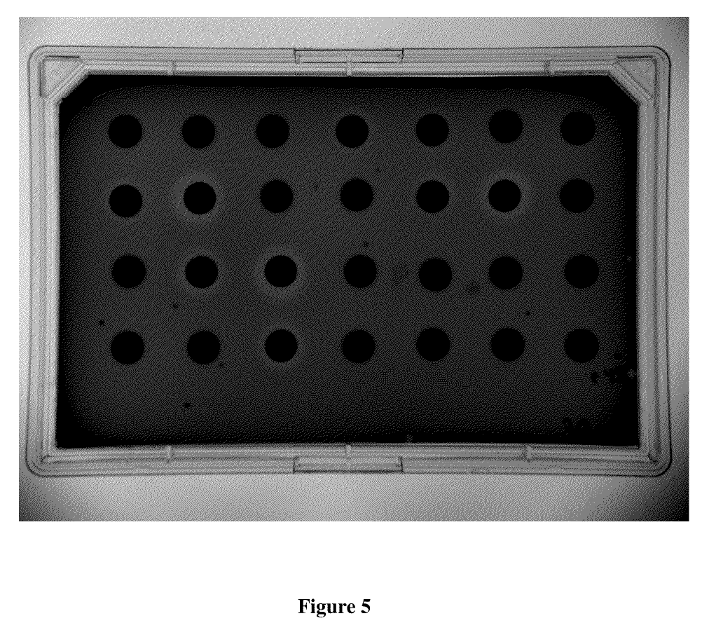

After ˜147 hours of total culture, 10% of the culture volume of each growth condition was processed and spotted on a blood-agar plate. A comparison of the hemolytic activity that is obtained with various concentrations of pure and crude glycerol is shown in FIG. 3.

As a further comparison, OKB105 D(upp)Spect^{R} cells grown in M9YE (6 g/L Na2HPO4, 3 g/L KH2PO4, 0.5 g/L NaCl, 1 g/L NH4Cl) with either 5 g/L glucose or 5 g/L crude glycerol. Once cell density dropped ˜10% from max OD650, cells were supplemented with 40 g/L tryptone. Cell free cultures were purified on seppak c18 columns, concentrated, and two dilutions of methanol (10 and 20 μL) were spotted on blood agar plate. As can be seen in FIG. 4, cells grown in glucose contain slightly larger halos than cells grown in crude glycerol.

Thus, both the growth curves (FIG. 2) and hemolytic data (FIG. 3) clearly show that crude glycerol and pure glycerol produce the same growth of strain OKB105 Δ(upp)Spect^{R}, as well as similar amount of hemolytic activity.

### Example 3

### Surfactin Production and Yield by Bacillus subtilis Grown on Crude Glycerol

The yield of surfactin produced by cells grown in media containing crude glycerol as a carbon source was determined. We first engineered a construct comprising a temperature inducible promoter Pr from the lambda bacteriophage that drives expression of surfactin. We obtained the plasmid pPR54 (G. Serrano-Heras et al., Plasmid, 54:278-282, 2005) and amplified two fragments from surfactin to replace the native promoter of surfactin with a temperature inducible promoter. The piece upstream of the surfactin promoter was obtained using primers:

UP-BstXI-SAlI-FW:

[SEQ ID NO: 1]

5′-GCAGCTCCATAGGATTGGTCGACGGCATTCTGATGATGAGGTCCGCT
TG-3′
and
UP-BstXI-BK:

[SEQ ID NO: 2]

5′-ATACATCTGCAGGCTTCAAGAATAGGCATGAGGCTTTCTC-3′.

The piece downstream of the surfactin promoter was amplified using primers:

DOWN-XbaI-FW-1:

[SEQ ID NO: 3]

5′-TTCTGCTCTAGATGTAGTACTTTGGGCTATTTCGGCTGTT-3′
and
DOWN-BstXI-BK:

[SEQ ID NO: 4]

5′-
CTGATACCACTCCTATGGAACCGCTGGATGATCAGAAAGCAGTTCAG-
3′.

Both pieces were digested with BstXI, cleaned with the PCR purification kit from Qiagen and ligated with T4 DNA ligase. The ligated product was amplified with primers DOWN-XbaI-FW and UP-BstXI-BK and digested with XbaI and PstI and cloned into pPR54 that was digested with the same enzymes. The ligation mixture was transformed into Bacillus subtilis 6GM (BGSC) and a plasmid with the desired sequenced was transformed into OKB105 Δ(upp)Spect^{R}. As a result of this transformation, the surfactin promoter was replaced with the lambda temperature inducible promoter. During transformation, the repressor of the temperature inducible promoter (cI857) was introduced into the Bacillus chromosome. The resulting strain was named OKB105 Δ(upp)Spect^{R} Δ(Psurf) Pr+ Phleo^{R}

A “scraping” from a frozen stock of OKB10 5 Δ(upp)Spect^{R} Δ(Psurf) Pr⁺ Phleo^{R} was inoculated into 25 ml M9YE containing 5 μl crude glycerol. This mixture was incubated at 30° C. for about 20 hrs. The following morning 2% of that culture was used to inoculate the same fresh media at 37° C. until the cells reached OD650 nm=1.0, then 9 ml were transferred to 50-ml conical tubes to which various amounts of glycerol, soy extract, amino acids (Leu, Val, Glu, and Asp), tryptone were added and then transferred to a 45° C. incubator to induce Pr. Cells were incubated for about 22 hrs. Supernatants were passed through a C18 column that was pre-washed with 100% methanol and then equilibrated with 0.1% TFA in water. Samples were loaded onto the columns and the bound material was washed with 40% methanol/0.1% TFA. Bound material was recovered by elution with 100% methanol and spotted onto blood agar plates to detect the amount of surfactin, as judged by the hemolytic activity present on the plates. The material added to 9-ml of cells is indicated in Table 2.

TABLE 2
Supplements to Cell Culture

| Sample | Supplement |
|---|---|
| 22916 | None |
| 22917 | None |
| 22918 | None |
| 22919 | 100 μl of 10% soy extract |
| 22920 | 300 μl of 10% soy extract |
| 22921 | 1000 μl of 10% soy extract |
| 22922 | 100 μl of 50% glycerol |
| 22923 | 100 μl of 10% soy extract + 100 μl of 50% glycerol |
| 22924 | 300 μl of 10% soy extract + 100 μl of 50% glycerol |
| 22925 | 1000 μl of 10% soy extract + 100 μl of 50% glycerol |
| 22926 | 200 μl of 50% glycerol |
| 22927 | 100 μl of 10% soy extract + 200 μl of 50% glycerol |
| 22928 | 300 μl of 10% soy extract + 200 μl of 50% glycerol |
| 22929 | 1000 μl of 10% soy extract + 200 μl of 50% glycerol |
| 22930 | 400 μl of 50% glycerol |
| 22931 | 100 μl of 10% soy extract + 400 μl of 50% glycerol |
| 22932 | 300 μl of 10% soy extract + 400 μl of 50% glycerol |
| 22933 | 1000 μl of 10% soy extract + 400 μl of 50% glycerol |
| 22934 | 200 μl of 50 mg/ml leucine |
| 22935 | 100 μl of 100 mg/ml glutamic acid |
| 22936 | 200 μl of 50 mg/ml aspartic acid |
| 22937 | 400 μl of 25 mg/ml valine |
| 22938 | 200 μl of 50 mg/ml leucine + 100 μl of 100 mg/ml glutamic |
| | acid + 200 μl of 50 mg/ml |
| | aspartic acid + 400 μl of 25 mg/ml valine |
| 22939 | 2 ml of 20% tryptone |
| 22940 | 2 ml of 10% soy extract |
| 22941 | None |
| 22942 | 90 ml of cells -> 9 ml of cells + 2 ml of 20% tryptone |
| 22943 | 90 ml of cells -> 9 ml of cells + 2 ml of 10% soy extract |

After 24 hrs, 10% of the volume was processed as described above using a C18 column. The hemolytic activity of the samples is shown in FIG. 5.

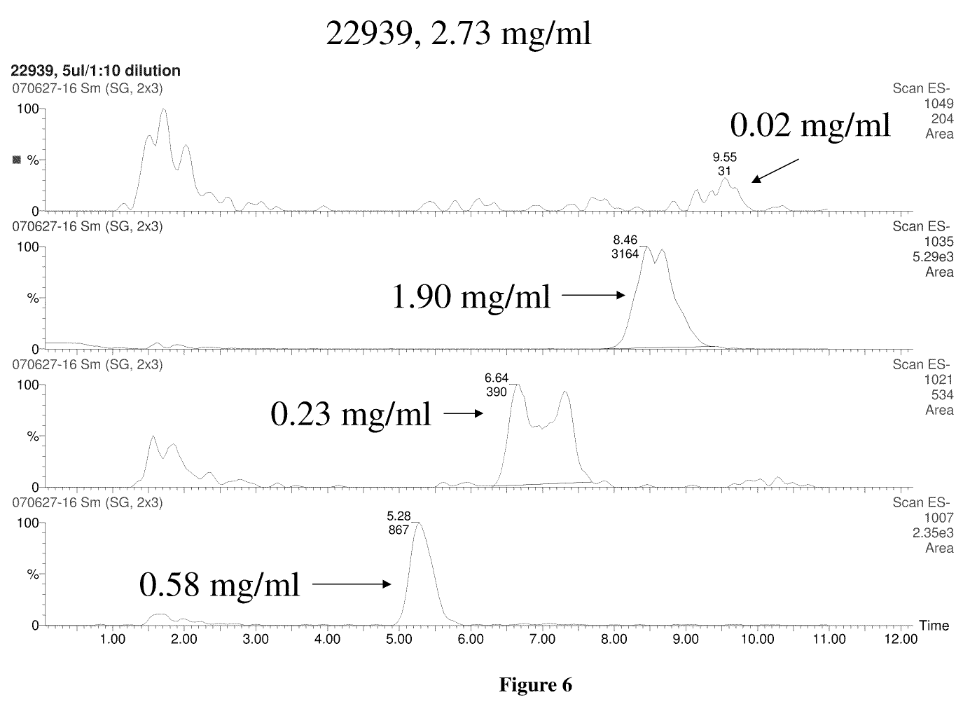

Following the hemolytic assay data, we selected those samples with the biggest halos (22916, 22919, 22920, 22923, 22924, 22927, 22928, 22931, 22932, 22939) to further investigate the actual amount of surfactin present in the media. Surfactin production was calculated by using LC-MS and calculating area under the curve compared to a known standard of surfactin that was purified to homogeneity, as judged by MS. Four m/z peaks (1007, 1021, 1035, 1049) were selected to quantify the titer of surfactin production. M/z peaks for sample 22939 are shown in FIG. 6.

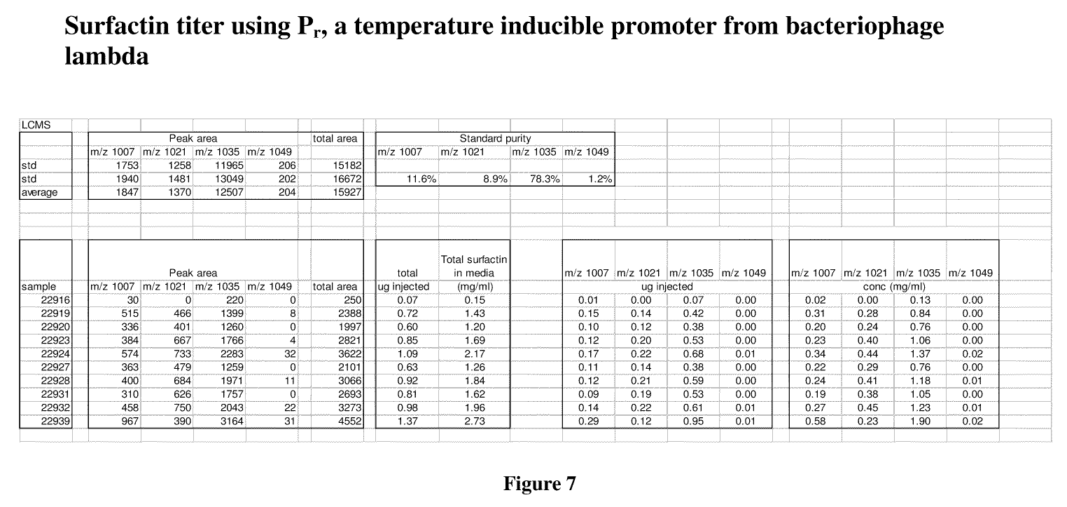

More than 95% of the standard, approximately 134 mg of purified surfactin, appeared to dissolve in methanol to produce a cloudy solution. After sonication an aliquot of this sample, it was diluted 1:10 in 90% methanol, 0.1% NH₄OAc and gave a clear solution. The samples were diluted 1:10 using the 90% methanol, 0.1% NH₄OAc. Both the samples and standard were run isocratically in water-acetonitrile-methanol-isopropanol (12.5:42.5:37.5:7.5) containing 0.075% NH₄OAc at a flow rate of 50 μl/min using a 1×100 mm ODS Hypersil with 3 μm particles and 120 Å pores (Agilent). The result of these experiments is shown in FIG. 7.

The total amount of surfactin present in the various media is given below in Table 3.

TABLE 3
Surfactin Present in Various Media

| Sample | Total surfactin in media (g/l) |
|---|---|
| 22916 | 0.15 |
| 22919 | 1.43 |
| 22920 | 1.20 |
| 22923 | 1.69 |
| 22924 | 2.17 |
| 22927 | 1.26 |
| 22928 | 1.84 |
| 22931 | 1.62 |
| 22932 | 1.96 |
| 22939 | 2.73 |

As shown in FIG. 6 and Table 3, the best surfactin titer achieved was 2.73 μl. Thus, the best yield of surfactin obtained (percent of crude glycerol converted to surfactin) was approximately 55%, which approaches the theoretical maximal yield of 60%.

### Example 4

### Surfactin Production in Bioreactors

Surfactin was produced by Bacillus subtilis strain OKB105 Δ(upp)Spect^{R} grown in 8 L bioreactors according to the conditions listed in Table 4 below. For comparison, Bacillus subtilis strain OKB105 Δ(upp)Spect^{R} was also grown in a 1 L shake flask.

TABLE 4
Bioreactor Conditions

| | | % Conversion for Total |
|---|---|---|
| | | Surfactin/Media |
| | 8 L Reactor Volume | component |
| Bioreactor 1 | | |
| Na2HPO4 = 6 g | 48 | 5.00 |
| KH2PO4 = 3 g | 24 | 10.00 |
| NaCl = 0.5 g | 4 | 60.00 |
| NH4Cl = 1 g | 8 | 30.00 |
| Yeast Extract = 3 g | 24 | 10.00 |
| Refined Glycerol = 5 g | 40 | 6.00 |
| Tryptone = 35.6 g | 284.8 | 0.84 |
| Bioreactor 2 | | |
| Na2HPO4 = 6 g | 48 | 5.83 |
| KH2PO4 = 3 g | 24 | 11.67 |
| NaCl = 0.5 g | 4 | 70.00 |
| NH4Cl = 1 g | 8 | 35.00 |
| Yeast Extract = 3 g | 24 | 11.67 |
| Refined Glycerol = 5.0 g | 40 | 7.00 |
| Tryptone = 10 g | 80 | 3.50 |
| | | % Conversion for Total |
| | | Surfactin/Media |
| Shake Flask | 1 L Reactor Volume | component |
| Na2HPO4 = 6 g | 6 | 16.67 |
| KH2PO4 = 3 g | 3 | 33.33 |
| NaCl = 0.5 g | 0.5 | 200.00 |
| NH4Cl = 1 g | 1 | 100.00 |
| Yeast Extract = 3 g | 3 | 33.33 |
| Refined Glycerol = | 6.25 | 16.00 |
| 6.25 g | | |
| Tryptone = 40 g | 40 | 2.50 |

Total surfactin production for the two bioreactors and the shake flask is shown in Table 5 below. Dry weights of surfactin are based on butanol extraction followed by vacuum concentration. Cells were collected by centrifugation, then dried by vacuum concentration. Media was collected as aqueous phase after butanol extraction, then dried by vacuum concentration.

TABLE 5
Total Surfactin Production

| | Shake | | |
|---|---|---|---|
| | Flask | Bioreactor 1 | Bioreactor 2 |
| reactor-surfactin (g/L) | 1 | 0.2 | 0.15 |
| foam from day 1-surfactin (g/L) | — | — | 0.23 |
| foam from day 5-surfactin (g/L) | — | 0.4 | 1.98 |
| total surfactin (g/L) | 1 | 0.6 | 2.13 |
| reactor volume (L) | 1 | 6 | 6.5 |
| foam volume (L) | — | 3 | 0.8 |
| reactor volume initial, without | 1 | 9 | 8 |
| evaporation (L) | | | |
| total surfactin (g/fermentation | 1 | 2.4 | 2.79 |
| volume, L) | | | |
| cells (g/L) | 3 | 4.9 | 2.1 |
| Media (g/L) | 56.4 | 53.7 | 27.8 |
| Media (g) total | 56.4 | 483.3 | 222.4 |
| % conversion (g/L) in reactor only | 1.77 | 0.37 | 0.54 |
| % conversion of entire | 1.77 | 0.50 | 1.25 |
| fermentation input media | | | |
| (foam + reactor) | | | |

As can be seen in Table 5, Bioreactor 1 yielded 2.4 g/l surfactin from 5 g/l refined glycerol, while Bioreactor 2 yielded 2.79 g/l surfactin from 5 g/l refined glycerol. Bioreactor 2 achieved a yield (percent of crude glycerol converted to surfactin) of approximately 56% (percent of refined glycerol converted to surfactin). Thus, like Example 3, the best yield of surfactin obtained in this Example approaches the theoretical maximal yield of 60%. This Example, in conjunction with Example 3, demonstrates that surfactin yields approaching the theoretical maximal yield can be obtained using media containing either refined glycerol or crude glycerol. Thus, crude glycerol has been demonstrated to be an advantageous carbon source for producing surfactin in Bacillus subtilis, and functions just as effectively as refined surfactin.

### Example 5

### Organic Acid Production by Saccharomyces Cerevisiae Grown on Crude Glycerol

Purification of raw materials prior to use in cultivation has often been necessary to remove contaminants that may inhibit microbial growth, such as salts and transesterification stabilizers. To date, there has not been any evidence that growth of S. cerevisiae could be supported by media supplemented with crude glycerol as the sole carbon and energy source.

Strains, plasmids, and media: The S. cerevisiae strain used was YPH-501 (ura3-52 lys2-801amber ade2-101ochre trp1-Δ63 his3-Δ200 leu2-Δ1) (Stratagene, La Jolla, Calif.). For general growth, YPAG media (1% yeast extract, 2% Bacto-peptone, 2% raw glycerol, 2% Bacto-agar, 0.0075% 1-adenine-hemisulfate salt) was used. For organic acid production, the growth media contained (g/L) raw glycerol 50.0, L-Methionine 0.25, NH₄Cl 1.8, KH₂PO₄ 2.0, MgSO₄.7H₂O 1.0, NaCl 0.6, CaCl₂.2H₂O 0.2, ZnSO₄.7H₂O 0.005 and yeast extract 1.0. The medium also contained 1 mL corn steep liquor (Sigma), 2 mL vitamin solution, and 2 mL solution of trace elements. The vitamin solution contains (mg/100 mL) biotin 2.0, calcium pantothenate 40.0, folic acid 2.0, inositol 200.0, niacin 40.0, p-aminobenzoic acid 20.0, pyridoxine hydrochloride 40.0, riboflavin 20.0, and thiamine hydrochloride 40.0. The solution of trace elements contains (mg/100 mL) H₃BO₃ 50.0, CuSO₄.5H₂O 4.0, KI 10.0, FeSO₄.7H₂O 20.0, MnSO₄.4H₂O 40.0, Na₂MoO₄.2H₂O 20.0, and ZnSO₄.7H₂O 40.0.

Cells were grown in 250 mL flasks containing 50 mL media at 30° C. with shaking. For organic acid production, 50 μl of the YP culture was used to inoculate acid growth media supplemented with the appropriate sugar and amino acid complementation and the cultures were grown as previously described. Cell density was monitored by measuring absorbance at 600 nm and by cell counts using a hemocytometer.

Gas Chromatography/Mass Spectrometry Analysis: 50 μl of each EtOH-precipitated sample was spiked with 0.5 μg of tropic acid as an internal standard and dried with nitrogen into a 1.0 ml reactivial. The tms derivative was formed by the addition of 50 μl of BSTFA-TMCS-pyridine (200:2:50) and heating at 80° C. for 1 hour. For GCMS, 1 μl was injected. An external standard mixture of the organic acids was run at the start of the analysis, after half the samples were injected and at the end of the analysis. Separation was performed on a DB5MS fused silica column (30 m×0.25 mm) with a temperature program from 70-172 degrees at 5 degrees per minute to 250 degrees at 50 degrees per minute with a 1 minute hold at 250 to clean the column. One injection of each sample was made. Samples were analyzed by positive electron impact ionization and selected ion monitoring for the ions specific to each organic acid. Data was analyzed by calculating ratios of the areas of the organic acids to the area of the internal standard and against the response of the external standard mix.

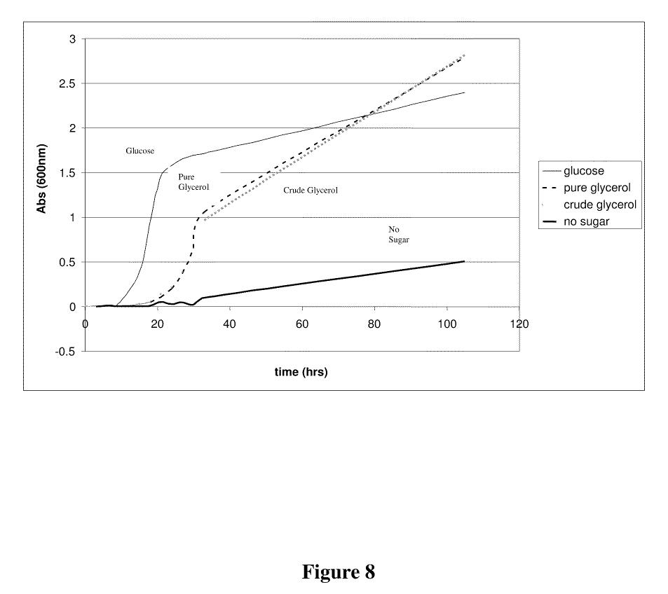

Results: Cultures of S. cerevisiae were initiated in a general YP media supplemented with either glucose, purified glycerol, raw glycerol, or no sugar to determine the extent of growth on glycerol as a sole carbon source. The culture grown on glucose demonstrated the first exponential growth phase at approximately 10 hours. Both glycerol cultures demonstrated parallel growth rates, entering exponential phase at approximately 20 hours. After 108 hours, it was demonstrated that yeast cultures grown on either glucose or glycerol were able to reach the same density in contrast to the culture lacking a sugar source, which failed to produce a significant amount of cells (see FIG. 8).

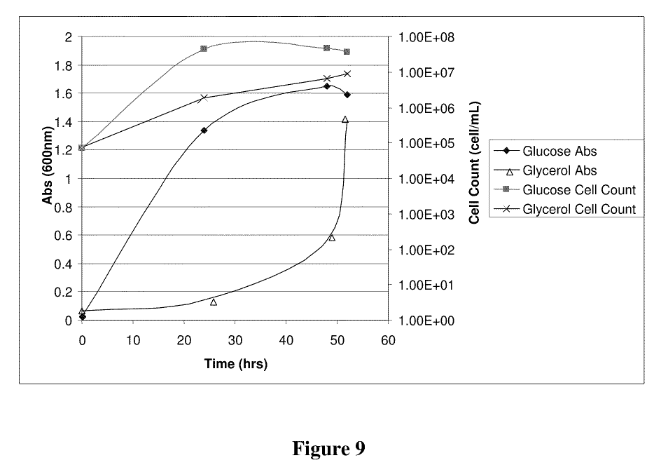

A saturated YPD culture of S. cerevisiae was used to inoculate nitrogen-limited acid production media supplemented with either glucose or raw glycerol. Absorbance measurements and cell counts at 0, 24 and 48 hours demonstrate that growth in the acid production cultures follow a similar trend to the YP cultures, with the glycerol-supplemented culture exhibiting a significant initial delay in growth relative to the glucose-supplemented culture but over time approaching comparable levels of density (see FIG. 9).

Samples of media were taken at 24 hrs and 48 hrs and subjected to GC/MS analysis to determine the level of organic acid production in each respective media (Table 6).

TABLE 6
Comparison of organic acid production in nitrogen-limited media
supplemented with either glucose or raw glycerol. Samples of each
culture were taken after 24 hrs incubation and subjected to GC/MS
analysis.

| | |
|---|---|
| Organic Acid | Conc. (μg/mL) |

Nitrogen-limited Glucose Media After 24 hrs Incubation

| | |
|---|---|
| Pyruvic Acid | 185.36 |
| Succinic Acid | 44.55 |
| Fumaric Acid | 1.07 |
| Malic Acid | 59.05 |
| Citric Acid | 30.73 |

Nitrogen-limited Raw Glycerol Media After 24 hrs Incubation

| | |
|---|---|
| Pyruvic Acid | 11.89 |
| Succinic Acid | 19.62 |
| Fumaric Acid | 0.25 |
| Malic Acid | 6.61 |
| Citric Acid | 6.09 |

The results indicate that the level of organic acid production supported by raw glycerol is lower than the level of organic acid production supported by glucose. The relative differences between the two media, however, are not consistent. The level of pyruvic acid demonstrates a 94% reduction, whereas the level of succinic acid demonstrates a 54% reduction. The percentage of reduction of the remaining organic acids fall between the two extremes.

Summary: The growth of S. cerevisiae in media supplemented with raw glycerol can achieve comparable levels of cell density relative to glucose given a sufficient amount of incubation time. This was demonstrated in enriched media, lacking any selective pressure, as well as nitrogen-limited, selective by amino acid complementation. In 2001, Lee and coworkers demonstrated that the level of glycerol metabolism in bacterial cultures of Anaerobiospirillum succiniciproducens was strongly dependent on the level of yeast extract in the culture medium (Lee et al., Succinic acid production with reduced by-product formation in the fermentation of Anaerobiospirillum succiniciproducens using glycerol as a carbon source, Biotechnology and Bioengineering 72(1): 41-48, 2001). These results suggest that with proper media modifications, S. cerevisiae cultures can be grown to high densities utilizing a more cost efficient carbon source. Although the level of organic acid production in raw glycerol media is globally lower than that of glucose media, the relative levels of individual organic acids are not constant. This suggests that a metabolic shift to glycerol utilization may result in a redistribution of carbon source among the organic acids. Under conditions used in this Example, the yeast appear to exhibit a tendency to favor production of succinic acid.

### Example 6

### Distillers Grains and Refined Glycerol can be Used in Combination to Produce Surfactin

Design: A culture of Bacillus subtilis containing the wild type surfactin promoter driving expression of surfactin synthetase was grown in LB media to saturation at 37° C. and an equal volume of this media was used to seed seven new cultures in the minimal salts media comprising 6 g/L Na₂HPO₄, 3 g/L KH₂PO₄, 0.5 g/L MgSO₄*7H₂O, 0.18 g/L CaCl₂*2H₂O, 0.025 g/L FeSO₄*7H₂O, 0.022 g/L MnCl₂*4H₂O, 1 g/L Yeast Extract, 1 g/L L-Tryptophan, and 1 g/L L-Arginine. The media also contained a Carbon Source (85 g/L Maltose or 170 g/L Maltose, either autoclaved directly with media or added as a filter sterilized liquid prior to culture; or 5 g/L refined glycerol added as an autoclaved liquid prior to culture) and a Nitrogen Source (16.5 g/L Distillers Grains or 33 g/L distillers grains) in seven different combinations as shown in Table 7.

TABLE 7
Media Combinations of Carbon Source and Nitrogen Source

| | 85 g/L | 170 g/L | 85 g/L | 170 g/L | |
|---|---|---|---|---|---|
| | maltose- | maltose- | maltose- | maltose- | 5 g/L |
| | filtered | filtered | autoclaved | autoclaved | glycerol |
| 33 g/L | Culture 2 | Culture 1 | Culture 4 | Culture 3 | Culture 7 |
| Distillers | | | | | |
| Grains | | | | | |
| 66 g/L | Culture 6 | Culture 5 | | | |
| Distillers | | | | | |
| Grains | | | | | |

These seven cultures were grown at 37° C. for 95.25 hours before being spun down and processed. 500 μL samples were purified on Harvard Apparatus c18 macro spin columns, eluted in an equal volume of methanol, and concentrated 5×. 15 μL of each concentrated sample was then spotted onto a blood agar plate for hemolytic analysis and halos were observed the following day. Mass spec analysis was also performed on these same concentrated samples for all seven culture combinations.

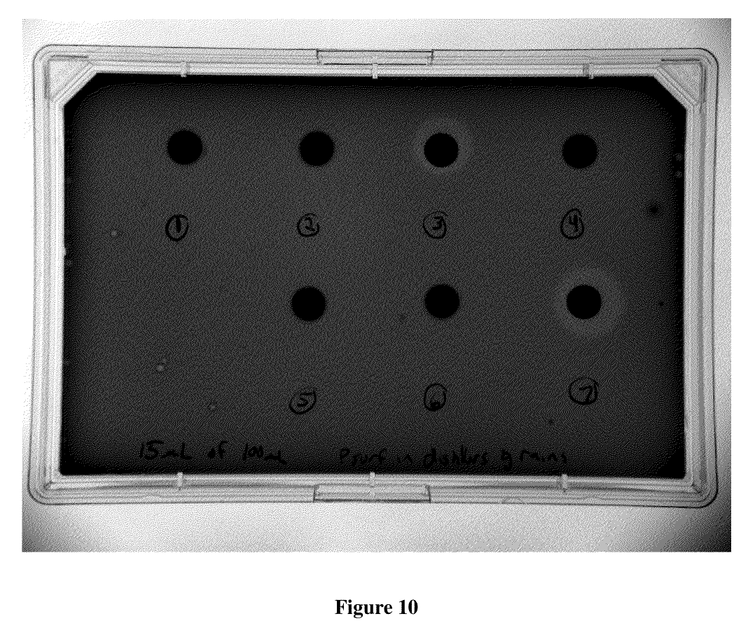

Results: As show in FIG. 10, hemolytic analysis (measured by hemolytic halo size) showed that Culture 7, containing 33 g/L distillers grains and 5 g/L refined glycerol, produced the most surfactin. Culture 3, containing 33 g/L distillers grains and 170 g/L Maltose (autoclaved in media), produced the second most surfactin. No other samples produced halos.

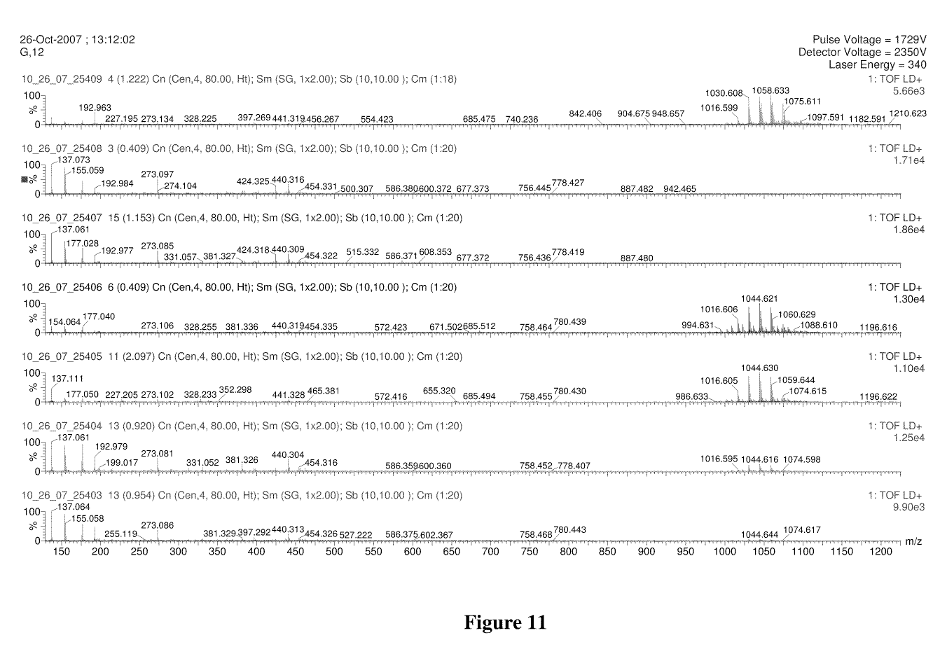

As show in FIG. 11, mass spectrometry analysis showed peaks for the same two conditions described above as well as peaks for 33 g/L distillers grains and 85 g/L Maltose (autoclaved in media), and 33 g/L distillers grains and 85 g/L Maltose (liquid added prior to culture).

This Example demonstrates that surfactin can be produced by using distillers grains as a nitrogen source and refined glycerol as a carbon source.

### Example 7

### Comparison of Distillers Grains Supplementation with Tryptone Supplementation

A culture of Bacillus subtilis containing the wild type surfactin promoter driving expression of surfactin synthetase was grown in Minimal Salts Media (6 g/L Na2HPO4, 3 g/L KH2PO4, 0.5 g/L NaCl, 1 g/L NH4Cl, 3 g/L Yeast Extract) and crude glycerol to saturation at 37° C. This culture was used to seed two new cultures using the same media to an OD650˜0.1. One of these cultures was moved to 37° C. and growth was monitored until the OD650 approached 0.5. The other culture was moved to 45° C. and growth was monitored until the OD650 approached 0.5. Both cultures were subsequently split off into 10 mL aliquots, supplemented with either 10 g/L autoclaved tryptone or 33 g/L filtered autoclaved distillers grains, and moved to either 30° C., 37° C. or 45° C. These cultures were grown for ˜46 and ˜70 hours before being spun down and processed. 500 μL samples were purified on Harvard Apparatus c18 macro spin columns, eluted in an equal volume of methanol, and concentrated 5×. 15 μL of each concentrated sample was then spotted onto a blood agar plate for hemolytic analysis and halos were observed the following day.

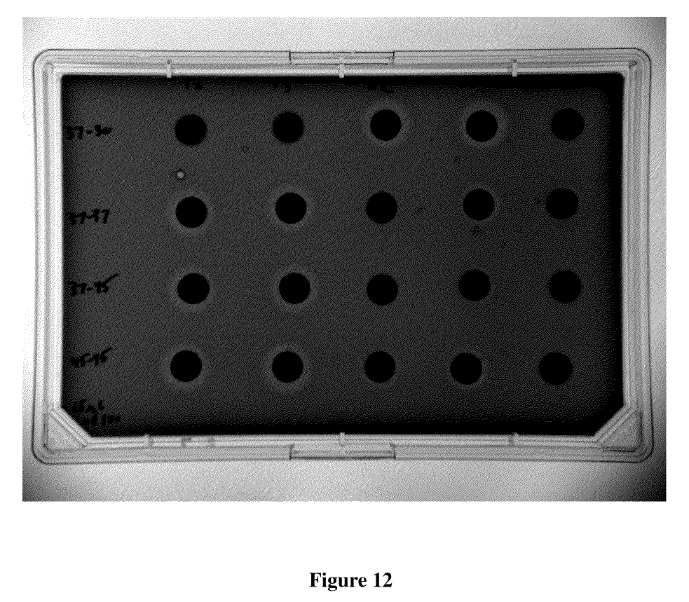

Results: As show in FIG. 12, hemolytic analysis (measured by hemolytic halo size) showed that cultures grown at 37° C. and shifted to 30° C. upon supplementation with distillers grains showed halos whereas supplementation with tryptone under the same conditions did not. Cultures grown at 37° C. and shifted to either 37° C. or 45° C. upon supplementation with tryptone showed larger halos than supplementation with distillers grains under the same conditions. Cultures grown at 45° C. and returned to 45° C. upon supplementation with tryptone showed larger halos than supplementation with distillers grains under the same conditions.

This Example demonstrates that distillers grains can be used for supplementation in combination with crude glycerol to produce halos comparable to tryptone supplementation. This Example further demonstrates that tryptone supplementation can be done while the cells are still in log phase as long as the cells are kept at 37° C. or moved to 45° C. Moreover, this Example demonstrates that distillers grains supplementation can be used to produce surfactin and this can be done at lower temperatures while cells are in log phase.

The foregoing description is to be understood as being representative only and is not intended to be limiting. Alternative methods and materials for implementing the invention and also additional applications will be apparent to one of skill in the art, and are intended to be included within the accompanying claims.

What is claimed is:

1. A cell culture medium for growing Bacillus subtilis, the cell culture medium comprising crude glycerol.

2. The cell culture medium of claim 1, wherein the cell culture medium substantially lacks refined glycerol.

3. The cell culture medium of claim 1, wherein the crude glycerol comprises:
about 80% glycerol;
about 3-10% fatty acid methyl esters;
less than about 1% methanol; and
about 7-16% water.

4. The cell culture medium of claim 1, wherein the cell culture medium comprises about 0.5% crude glycerol.

5. The cell culture medium of claim 4, wherein the crude glycerol is autoclaved and filtered.

6. The cell culture medium of claim 5, further comprising Na₂HPO₄, KH₂PO₄, NaCl, and NH₄Cl.

7. The cell culture medium of claim 6, wherein the cell culture medium comprises:
Na₂HPO₄ at a concentration of about 6 g/L;
KH₂PO₄ at a concentration of about 3 g/L;
NaCl at a concentration of about 0.5 g/L; and
NH₄Cl at a concentration of about 1 g/L.

8. The cell culture medium of claim 1, further comprising a nitrogen source selected from the group consisting of: total soy extract, tryptone, yeast extract, casamino acids, distiller grains, and combinations thereof.

9. A method for growing Bacillus subtilis in a cell culture, comprising growing the microorganism in a cell culture medium comprising crude glycerol, wherein the cell culture medium substantially lacks refined glycerol.

10. The method of claim 9, wherein the Bacillus subtilis produces a lipopeptide.

11. The method of claim 10, wherein the lipopeptide comprises surfactin.

12. The method of claim 11, wherein the yield of surfactin produced from the crude glycerol is at least about 25%.

13. The method of claim 12, further comprising the step of providing a nitrogen source after growth of the microorganism has been initiated,
which nitrogen source is selected from the group consisting of: tryptone, total soy extract, yeast extract, casamino acids, distiller grains, and combinations thereof;
wherein the microorganism produces surfactin to an increased level relative to the level of surfactin that is produced by a microorganism grown under otherwise identical conditions in an otherwise identical cell culture medium that lacks the provided nitrogen source.

14. The method of claim 13, wherein the nitrogen source is added during stationary phase.

15. The method of claim 14, wherein the nitrogen source is not added during growth phase.

16. The method of claim 15, wherein the cell culture is grown at a relatively low temperature or within a relatively low temperature range.

17. The method of claim 16, wherein the relatively low temperature is about 30° C.

18. The method of claim 13, wherein the nitrogen source is added during growth phase.

19. The method of claim 18, wherein the nitrogen source comprises distillers grains and further wherein the cell culture is grown at a relatively low temperature or within a relatively low temperature range.

20. The method of claim 19, wherein the relatively low temperature is about 30° C.

21. The method of claim 18, wherein the cell culture is grown at a relatively high temperature or within a relatively high temperature range.

22. The method of claim 21, wherein relatively high temperature is about 37° C.

23. The method of claim 21, wherein relatively high temperature is about 45° C.

24. A method for growing Saccharomyces cerevisiae in a cell culture, comprising growing the microorganism in a cell culture medium comprising crude glycerol, wherein the cell culture medium substantially lacks refined glycerol.

25. The method of claim 24, wherein the Saccharomyces cerevisiae produces an organic acid.

26. The method of claim 25, wherein the Saccharomyces cerevisiae produces an organic acid selected from the group consisting of pyruvic acid, succinic acid, fumaric acid, malic acid, maleic acid, citric acid and combinations thereof.

27. The method of claim 25, wherein the Saccharomyces cerevisiae produces an organic acid to a level of about 5 μg/mL.

28. The method of claim 24, wherein cell density of the cell culture is at least 75% the cell density of a second cell culture grown under otherwise identical conditions in an otherwise identical cell culture medium that lacks crude glycerol.
